(19) United States
(12) Patent Application Publication
(10) Pub. No.: US 2023/0091156 A1
(43) Pub. Date: Mar. 23, 2023

(54) METHODS AND MATERIALS FOR ASSESSING AND TREATING ARTHRITIS

(71) Applicant: Mayo Foundation for Medical Education and Research, Rochester, MN (US)

(72) Inventors: Jaeyun Sung, Rochester, MN (US); John M. Davis, III, Rochester, MN (US); Benjamin Hur, Rochester, MN (US)

(21) Appl. No.: 17/899,272

(22) Filed: Aug. 30, 2022

Related U.S. Application Data
(60) Provisional application No. 63/243,933, filed on Sep. 14, 2021.

(51) Int. Cl. A61K 31/525; A61K 31/47; A61K 31/4402; A61K 31/415; A61P 19/02

(52) U.S. Cl. CPC A61K 31/525; A61K 31/47; A61K 31/4402; A61K 31/415; A61P 19/02

(57) ABSTRACT

This document provides methods and materials for assessing and/or treating mammals (e.g., humans) having arthritis (e.g., rheumatoid arthritis). For example, the presence of distinct metabolite signatures in a sample obtained from a mammal (e.g., a human) having arthritis (e.g., rheumatoid arthritis) can be used to determine the disease activity status of the arthritis. Also provided are materials and methods for treating mammals (e.g., a human) having arthritis (e.g., rheumatoid arthritis).

### CROSS-REFERENCE TO RELATED APPLICATIONS

This application claims the benefit of U.S. Patent Application Ser. No. 63/243,933, filed on Sep. 14, 2021. The disclosure of the prior application is considered part of (and is incorporated by reference in) the disclosure of this application

### TECHNICAL FIELD

This document relates to methods and materials for assessing and/or treating mammals (e.g., humans) having arthritis (e.g., rheumatoid arthritis). For example, the presence of a distinct metabolite signature in a sample obtained from a mammal (e.g., a human) having arthritis (e.g., rheumatoid arthritis) can be used to determine the disease activity status of the arthritis. Also provided are materials and methods for treating mammals (e.g., a human) having arthritis (e.g., rheumatoid arthritis).

### BACKGROUND INFORMATION

Rheumatoid arthritis (RA) is a chronic, autoimmune inflammatory disease primarily affecting the small diarthrodial joints and other organ systems (Guo et al., Bone Res., (1):1-14 (2018); Aletaha et al., Arthritis & Rheumatism, 62(9):2569-2581 (2010); Smolen et al., Nature Reviews Disease Primers, 4(1):18001 (2018); and Firestein, Nature, 423(6937):356-361 (2003)) that can eventually lead to bone/cartilage erosion, joint deformity, loss in mobility, and organ damage (Aletaha et al., JAMA, 320(13):1360-1372 (2018)). Known to be associated with a variety of factors, such as genetic susceptibility, age, sex, smoking status, and dietary habits, RA is diagnosed in nearly 5 per 1000 adults worldwide, and women are 2 to 3 times more likely to develop RA than men (Aletaha et al., JAMA, 320(13):1360-1372 (2018)).

### SUMMARY

This document provides methods and materials for assessing and/or treating mammals (e.g., humans) having arthritis (e.g., RA). In some cases, this document provides methods and materials for determining the disease activity status of a mammal (e.g., a human) having arthritis (e.g., RA) based, at least in part, on the metabolite signature in a sample (e.g., a blood sample) obtained from the mammal. For example, a sample (e.g., a blood sample) obtained from a mammal having arthritis (e.g., RA) can be assessed to determine the disease activity status of the RA based, at least in part, on the presence or absence of an altered level (e.g., an increased level or a decreased level) of 15 or more (e.g., 18, 20, 22, 30, 40, 50, 51, or more) metabolites (e.g., circulating metabolites) in the sample. As demonstrated herein, distinct plasma metabolite signatures can be associated with RA disease activity status. Having the ability to determine the metabolite signature of a mammal (e.g., a human) having arthritis (e.g., rheumatoid arthritis) using a blood sample provides unique and non-invasive methods for determining disease activity status that is quicker and more cost-effective that current methods.

In general, one aspect of this document features a method for assessing a mammal having arthritis. The method comprises (or consists essentially of, or consists of) (a) determining if a blood sample from the mammal comprises a low disease activity signature or a moderate-to-high disease activity signature, (b) classifying the mammal as having low disease activity if the blood sample comprises the low disease activity signature, and (c) classifying the mammal as having moderate-to-high disease activity if the blood sample comprises the moderate-to-high disease activity signature, wherein the low disease activity signature comprises (1a) an increased level of five or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of five or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate, and wherein the moderate-to-high disease signature comprises (2a) an increased level of five or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of five or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The mammal can be a human. The arthritis can be a rheumatoid arthritis. The blood sample can be a plasma sample. The method can comprise classifying the mammal as having low disease activity. The low disease activity signature can comprise (1a) an increased level of or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of 10 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The low disease activity signature can comprise (1a) an increased level of 15 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of 15 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The low disease activity signature can comprise (1a) an increased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The low disease activity signature can comprise (1a) an increased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol and (1b) a decreased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The method can comprise classifying the mammal as having moderate-to-high disease activity. The moderate-to-high disease signature can comprise (2a) an increased level of 10 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of 10 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The moderate-to-high disease signature can comprise (2a) an increased level of 15 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of 15 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The moderate-to-high disease signature can comprise (2a) an increased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The moderate-to-high disease signature can comprise (2a) an increased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate and (2b) a decreased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol.

In another aspect, this document features a method for treating a mammal having arthritis. The method comprises (or consists essentially of, or consists of) (a) determining that a blood sample from the mammal comprises a low disease activity signature, and (b) administering an arthritis drug to the mammal, wherein the low disease activity signature comprises (1a) an increased level of five or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of five or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The mammal can be a human. The arthritis can be a rheumatoid arthritis. The blood sample can be a plasma sample. The low disease activity signature can comprise (1a) an increased level of 10 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of 10 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The low disease activity signature can comprise (1a) an increased level of 15 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of 15 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The low disease activity signature can comprise (1a) an increased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The low disease activity signature can comprise (1a) an increased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol and (1b) a decreased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The arthritis drug can be selected from the group consisting of methotrexate, hydroxychloroquine, sulfasalazine, and leflunomide.

In another aspect, this document features a method for treating a mammal having arthritis. The method comprises (or consists essentially of, or consists of) administering an arthritis drug to a mammal that was identified as having blood or plasma comprises a low disease activity signature, wherein the low disease activity signature comprises (1a) an increased level of five or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of five or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The mammal can be a human. The arthritis can be a rheumatoid arthritis. The low disease activity signature can comprise (1a) an increased level of 10 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of 10 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The low disease activity signature can comprise (1a) an increased level of 15 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of 15 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The low disease activity signature can comprise (1a) an increased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The low disease activity signature can comprise (1a) an increased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol and (1b) a decreased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate. The arthritis drug can be selected from the group consisting of methotrexate, hydroxychloroquine, sulfasalazine, and leflunomide.

In another aspect, this document features a method for treating a mammal having arthritis. The method comprises (or consists essentially of, or consists of) (a) determining that a blood sample from the mammal comprises a moderate-to-high disease activity signature, and (b) administering an arthritis drug to the mammal or performing surgery to treat the arthritis, wherein the moderate-to-high disease signature comprises (2a) an increased level of five or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of five or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The mammal can be a human. The arthritis can be a rheumatoid arthritis. The blood sample can be a plasma sample. The moderate-to-high disease signature can comprise (2a) an increased level of 10 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of 10 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The moderate-to-high disease signature can comprise (2a) an increased level of 15 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of 15 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The moderate-to-high disease signature can comprise (2a) an increased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The moderate-to-high disease signature can comprise (2a) an increased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate and (2b) a decreased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The method can comprise administering the arthritis drug to the mammal. The arthritis drug can be selected from the group consisting of adalimumab, certolizumab, etanercept, golimumab, infliximab, abatacept, tocilizumab, sarilumab, rituximab, tofacitinib, baricitinib, and upadacitinib. The method can comprise performing the surgery.

In another aspect, this document features a method for treating a mammal having arthritis. The method comprises (or consists essentially of, or consists of) administering an arthritis drug to a mammal or performing surgery on the mammal, wherein the mammal was identified as having blood or plasma comprises a moderate-to-high disease activity signature, wherein the moderate-to-high disease signature comprises (2a) an increased level of five or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of five or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The mammal can be a human. The arthritis can be a rheumatoid arthritis. The moderate-to-high disease signature can comprise (2a) an increased level of 10 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of 10 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The moderate-to-high disease signature can comprise (2a) an increased level of 15 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of 15 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The moderate-to-high disease signature can comprise (2a) an increased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The moderate-to-high disease signature can comprise (2a) an increased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate and (2b) a decreased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol. The method can comprise administering the arthritis drug to the mammal. The arthritis drug can be selected from the group consisting of adalimumab, certolizumab, etanercept, golimumab, infliximab, abatacept, tocilizumab, sarilumab, rituximab, tofacitinib, baricitinib, and upadacitinib. The method can comprise performing the surgery.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. A multi-approach discovery strategy to identify metabolites indicative of RA disease activity. (A) Differentially abundant metabolites between higher and lower disease activity groups were identified using a mixed-effects logistic regression model adjusted for patient age and sex, as well as for Patient ID to control for having multiple samples from the same patient. (B) A selection scheme to identify metabolites associated with DAS28-CRP. Metabolites were selected with mixed-effects linear regression. To further demonstrate their association with DAS28-CRP, these metabolites were used to construct a generalized linear model for predicting DAS28-CRP. Predictive performance of the model was evaluated on the discovery cohort (using a cross-validation technique) and on a validation cohort.

FIG. 2. Plasma metabolites differentiating between higher and lower disease activity groups in RA. A total of 2 and 31 metabolites were found to be significantly increased in higher (DAS28-CRP>3.2, n=52) and lower (DAS28-CRP≤3.2, n=76) disease activity groups, respectively. Each point corresponds to a metabolite (686 total). Differentially abundant metabolites were found using a mixed-effects logistic regression model on the discovery cohort (128 samples), for which age and sex were adjusted. Metabolites with P-value <0.05 (based upon the corresponding coefficient of the regression model) were considered as significantly different between groups. P-values and fold-changes for all metabolites are listed in Table 2. Metabolites in bold have been previously described in the literature for their associations with RA.

FIG. 3. Evaluation of DAS28-CRP predictive performance in cross-validation. A modified leave-one-out cross-validation approach was used on the samples of the training group (128 samples) to test the performance of a generalized linear model (GLM) in predicting DAS28-CRP scores from metabolite abundances. Distributions of absolute errors from models with and without a feature selection scheme were compared to identify the more robust model. The GLM with the feature selection scheme performed better (MAE±SD: 1.51±1.89) than the model without feature selection (MAE±SD: 2.02±2.52).

FIG. 4A-B. GLM with feature selection provides improved DAS28-CRP prediction accuracy in an independent validation group (12 samples). (A) Performance of GLMs in predicting quantitative disease activity were evaluated on samples of an independent validation group. Distributions of absolute errors from models with and without a feature selection scheme were compared to identify the more robust model. (B) Selection of metabolic features prior to model training resulted in higher predictive performance, as evidenced by the stronger correlation between observed and predicted DAS28-CRPs. Three samples predicted to have negative DAS28-CRP values are omitted from the scatter-plot. Dashed violet line indicates ‘y=x’, i.e., an exact match between the observed and predicted values. 95% confidence interval for ρ with feature selection: [0.18, 0.90]; without feature selection: [−0.44, 0.68].

FIG. 5. Venn diagram of all plasma metabolites identified through the multi-approach discovery strategy. A total of 67 unique metabolites were identified, among which were found to have no association with the use of treatment. Notably, eight metabolites (6-bromotryptophan, bilirubin (E,E), biliverdin, glucuronate, N-acetyltryptophan, N-acetyltyrosine, serine, and trigonelline) in bold were not only consistently detected across both analytic approaches, but also found to have no association with any treatment use. Colored circles indicate metabolites whose abundances associate with treatment use. Metabolites with red triangles were found to have increasing abundances with worsening disease activity, whereas metabolites with blue triangles were found to have decreasing abundances with worsening disease activity.

FIG. 6A-B. Metabolites differentially abundant between two CRP patient groups. Among the 67 total metabolites identified through our multi-approach analysis on the discovery cohort (n=128), eight metabolites were identified to have significant associations with CRP group while controlling for confounding variables (regression coefficient for CRP, P<0.05) (A) Metabolites with higher abundances in the high-CRP group: mannose, beta-hydroxyisovalerate, (14 or 15)-methylpalmitate (a17:0 or i17:0), erucate (22:1n9), 10-undecenoate (11:1n1), and N-acetylcitrulline. (B) Metabolites with higher abundances in the low-CRP group: serine and linoleoylcarnitine (C18:3).

FIG. 7. Histogram of DAS28-CRPs corresponding to the 128 samples of the discovery cohort.

### DETAILED DESCRIPTION

This document provides methods and materials for assessing and/or treating mammals (e.g., humans) having arthritis (e.g., rheumatoid arthritis). In some cases, this document provides methods and materials for determining the disease activity status of a mammal having arthritis (e.g., RA), and, optionally, treating the mammal. For example, a sample obtained from a mammal having arthritis (e.g., RA) can be assessed to determine the disease activity status of the arthritis based, at least in part, on the presence or absence of an altered level (e.g., an increased level or a decreased level) of 15 or more metabolites (e.g., circulating metabolites) in the sample. As demonstrated herein, a distinct metabolite signature is present in mammals having low disease activity (e.g., having a disease activity−28 using C-reactive protein (DAS28-CRP) score of about 3.2 or less) and in mammals having moderate-to-high disease activity (e.g., having a DAS28-CRP score greater than about 3.2.

Any type of mammal can be assessed and/or treated as described herein. Examples of mammals that can have arthritis (e.g., RA) and that can be assessed and/or treated as described herein include, without limitation, primates (e.g., humans and monkeys), dogs, cats, horses, cows, pigs, sheep, rabbits, mice, and rats. In some cases, a human can be assessed and/or treated as described herein.

Rheumatoid arthritis, when present, can be in any appropriate joint within a mammal being assessed and/or treated as described herein. Examples of joints that can be arthritic in a mammal (e.g., a human) having rheumatoid arthritis include, without limitation, joints in the feet, joints in the hands, joints in the hips, joints in the knees, joints in the wrist, joints in the elbow, joints in the shoulder, and joints in the ankles.

Any appropriate method can be used to identify a mammal as having arthritis (e.g., RA). In some cases, laboratory tests (e.g., analysis of body fluids such as blood, urine, and/or joint fluid for, for example, biomarkers such as rheumatoid factor and anti-cyclic citrullinated protein (CCP) antibodies), imaging techniques (e.g., X-ray, computerized tomography (CT), and magnetic resonance imaging (MRI), and ultrasound) can be used to identify mammals (e.g., humans) as having arthritis (e.g., RA).

Once identified as being having arthritis (e.g., RA), a mammal can be assessed to determine the disease activity of the arthritis. For example, a sample (e.g., a blood sample) obtained from the mammal can be assessed for the presence, absence, or level of 15 or more metabolites (e.g., circulating metabolites). As described herein, a distinct metabolite signature in a sample obtained from a mammal having arthritis (e.g., RA) can be used to determine the disease activity of the arthritis.

Any appropriate sample from a mammal (e.g., a human) having arthritis (e.g., RA) can be assessed as described herein. In some cases, a sample can be a biological sample. In some cases, a sample can contain metabolites (e.g., amino acids, cofactors, vitamins, nucleotides, lipids, peptides, xenobiotics, and carbohydrates). Examples of samples that can be assessed as described herein include, without limitation, blood samples, whole blood samples, serum samples, and plasma samples. In some cases, plasma samples obtained from a mammal (e.g., a human) having arthritis (e.g., RA) can be assessed as described herein.

In some cases, a mammal (e.g., a human) having arthritis (e.g., RA) can be identified as having low disease activity (DAS28-CRP≤3.2) or moderate-to-high disease activity (DAS28-CRP>3.2) based, at least in part, on the presence of an altered level of 15 or more (e.g., 15, 18, 20, 22, 30, 40, 50, 51, or more) metabolites (e.g., circulating metabolites) in a sample (e.g., a plasma sample) obtained from the mammal. In some cases, an altered level of a metabolite can be an increased level of the metabolite. The term “increased level” as used herein with respect to a level of a metabolite refers to any level that is higher than a reference level of the metabolite. In some cases, an altered level of a metabolite can be a decreased level of the metabolite. The term “decreased level” as used herein with respect to a level of a metabolite refers to any level that is lower than a reference level of the metabolite. The term “reference level” for a particular metabolite refers to the median level of that metabolite present in samples obtained from a population of mammals (e.g., a population of 20, 50, 100, or more mammals), where a number of those mammals (e.g., about half of those mammals or about 40-60 percent of those mammals) have arthritis (e.g., RA) with low disease activity and a number of those mammals (e.g., the other about half or about 40-60 percent) have arthritis (e.g., RA) with moderate-to-high disease activity. For example, the term “reference level” for a particular metabolite refers to the median level of that metabolite present in samples obtained from a population of mammals (e.g., a population of 20, 50, 100, or more mammals), where 40 percent of those mammals have arthritis (e.g., RA) with low disease activity and 60 percent of those mammals have arthritis (e.g., RA) with moderate-to-high disease activity. In another example, the term “reference level” for a particular metabolite refers to the median level of that metabolite present in samples obtained from a population of mammals (e.g., a population of 20, 50, 100, or more mammals), where 60 percent of those mammals have arthritis (e.g., RA) with low disease activity and 40 percent of those mammals have arthritis (e.g., RA) with moderate-to-high disease activity. In some cases, abundance values, which are measured from a population of mammals, (or relative abundance values, which are measured and normalized from a population of mammals) that are set to differentiate between low disease activity and moderate-to-high disease activity as described herein can be used as a reference level. Examples of reference levels of particular metabolites for human plasma samples are set forth in Tables A and B. It will be appreciated that levels of metabolites from comparable samples are used when determining whether or not a particular level is an altered level.

TABLE A
Abundance values of 51 metabolites for human plasma
samples (measured by metabolon UPLC-MS/MS).

| | | | Abundance |
|---|---|---|---|
| | | Abundance | differences^{β} in |
| | | differences^{β} in low | moderate-high |
| Metabolite | Abundance value^{α} | disease activity | disease activity |
| isoursodeoxycholate | 1340080 | Increased (e.g., +9.70%). | Decreased (e.g., −13.64%). |
| | | In some cases, an | In some cases, a |
| | | increase of this | decrease of this |
| | | metabolite of 1, 2, 3, 4, | metabolite of 1, 2, 3, 4, |
| | | 5, 6, 7, 8, 9, or more | 5, 6, 7, 8, 9, 10, 11, 12, |
| | | percent as compared to | 13, or more percent as |
| | | a reference level can | compared to a |
| | | be used to identity low | reference level can be |
| | | activity disease. | used to identify |
| | | | moderate to high |
| | | | activity disease. |
| linoleoylcarnitine (C18:2) | 10125123.5 | Increased (e.g., +5.49%). | Decreased (e.g., −3.80%). |
| | | In some cases, an | In some cases, a |
| | | increase of this | decrease of this |
| | | metabolite of 1, 2, 3, 4, | metabolite of 1, 2, 3, |
| | | 5, or more percent as | or more percent as |
| | | compared to a | compared to a |
| | | reference level can be | reference level can be |
| | | used to identify low | used to identify |
| | | activity disease. | moderate to high |
| | | | activity disease. |
| dihomo-linoleoylcarnitine | 341755 | Increased (e.g., +6.28%). | Decreased (e.g., −7.63%) |
| (C20:2) | | In some cases, an | In some cases, a |
| | | increase of this | decrease of this |
| | | metabolite of 1, 2, 3, 4, | metabolite of 1, 2, 3, 4, |
| | | 5, 6, or more percent | 5, 6, 7, or more percent |
| | | as compared to a | as compared to a |
| | | reference level can be | reference level can be |
| | | used to identify low | used to identify |
| | | activity disease. | moderate to high |
| | | | activity disease. |
| N-acetyltyrosine | 191301 | Increased (e.g., +4.53%). | Decreased (e.g., −8.07%). |
| | | In some cases, an | In some cases, a |
| | | increase of this | decrease of this |
| | | metabolite of 1, 2, 3, 4, | metabolite of 1, 2, 3, 4, |
| | | or more percent as | 5, 6, 7, 8, or more |
| | | compared to a | percent as compared to |
| | | reference level can be | a reference level can |
| | | used to identify low | be used to identify |
| | | activity disease. | moderate to high |
| | | | activity disease. |
| (14 or 15)-methylpalmitate | 23250337 | Decreased (e.g., −2.92%). | Increased (e.g., +4.29%). |
| (a17:0 or i17:0) | | In some cases, a | In some cases, an |
| | | decrease of this | increase of this |
| | | metabolite of 1, 1.5, 2, | metabolite of 1, 2, 3, 4, |
| | | 2.5, or more percent as | or more percent as |
| | | compared to a | compared to a |
| | | reference level can be | reference level can be |
| | | used to identify low | used to identify |
| | | activity disease. | moderate to high |
| | | | activity disease. |
| 3-methylhistidine | 6881044 | 0.00% | Increased (e.g., +0.02%). |
| | | | In some cases, an |
| | | | increase of this |
| | | | metabolite of 0.005, |
| | | | 0.01, 0.015, or more |
| | | | percent as compared to |
| | | | a reference level can |
| | | | be used to identify |
| | | | moderate to high |
| | | | activity disease. |
| 1,6-anhydroglucose | 1731268 | Decreased (e.g., −2.40%). | Increased (e.g., +1.02%). |
| | | In some cases, a | In some cases, an |
| | | decrease of this | increase of this |
| | | metabolite of 1, 1.5, 2, | metabolite of 0.25, 0.5, |
| | | 2.3, or more percent as | 0.75, or more percent |
| | | compared to a | as compared to a |
| | | reference level can be | reference level can be |
| | | used to identify low | used to identify |
| | | activity disease. | moderate to high |
| | | | activity disease. |
| 1-methylhistidine | 648415 | Increased (e.g., +1.13%). | Decreased (e.g., −0.63%). |
| | | In some cases, an | In some cases, a |
| | | increase of this | decrease of this |
| | | metabolite of 0.25, 0.5, | metabolite of 0.3, 0.4, |
| | | 0.75, 1, or more | 0.5, or more percent as |
| | | percent as compared to | compared to a |
| | | a reference level can | reference level can be |
| | | be used to identify low | used to identify |
| | | activity disease. | moderate to high |
| | | | activity disease. |
| N-acetylneuraminate | 1096063.5 | Decreased (e.g., −0.12%). | Increased (e.g., +0.11%). |
| | | In some cases, a | In some cases, an |
| | | decrease of this | increase of this |
| | | metabolite of 0.05, | metabolite of 0.05, |
| | | 0.075, 0.085, 0.095, or | 0.075, 0.95, or more |
| | | more percent as | percent as compared to |
| | | compared to a | a reference level can |
| | | reference level can be | be used to identify |
| | | used to identify low | moderate to high |
| | | activity disease. | activity disease. |
| 4-guanidinobutanoate | 825106 | Increased (e.g., +7.15%). | Decreased (e.g., −3.63%). |
| | | In some cases, an | In some cases, a |
| | | increase of this | decrease of this |
| | | metabolite of 1, 2, 3, 4, | metabolite of 1, 2, 2.5, |
| | | 5, 6, 7, or more percent | 3, 3.5, or more percent |
| | | as compared to a | as compared to a |
| | | reference level can be | reference level can be |
| | | used to identify low | used to identify |
| | | activity disease. | moderate to high |
| | | | activity disease. |
| hypoxanthine | 47766902 | Decreased (e.g., −17.11%). | Increased (e.g. +14.38%). |
| | | In some cases, a | In some cases, an |
| | | decrease of this | increase of this |
| | | metabolite of 1, 2, 3, 4, | metabolite of 1, 2, 3, 4, |
| | | 5, 6, 7, 8, 9, 10, 11, 12, | 5, 6, 7, 8, 9, 10, 11, 12, |
| | | 13, 14, 15, 16, 17, or | 13, 14, or more percent |
| | | more percent as | as compared to a |
| | | compared to a | reference level can be |
| | | reference level can be | used to identify |
| | | used to identify low | moderate to high |
| | | activity disease. | activity disease. |
| 1-carboxyethylleucine | 117997 | Decreased (e.g., −6.09%). | Increased (e.g., +8.88%). |
| | | In some cases, a | In some cases, an |
| | | decrease of this | increase of this |
| | | metabolite of 1, 2, 3, 4, | metabolite of 1, 2, 3, 4, |
| | | 5, 6, or more percent | 5, 6, 7, 8, or more |
| | | as compared to a | percent as compared to |
| | | reference level can be | a reference level can |
| | | used to identify low | be used to identify |
| | | activity disease. | moderate to high |
| | | | activity disease. |
| ectoine | 980789 | Decreased (e.g., −6.06%). | Increased (e.g., +8.91%). |
| | | In some cases, a | In some cases, an |
| | | decrease of this | increase of this |
| | | metabolite of 1, 2, 3, 4, | metabolite of 1, 2, 3, 4, |
| | | 5, 6, or more percent | 5, 6, 7, 8, or more |
| | | as compared to a | percent as compared to |
| | | reference level can be | a reference level can |
| | | used to identify low | be used to identify |
| | | activity disease. | moderate to high |
| | | | activity disease. |
| lysine | 334960560 | Increased (e.g., +2.12%). | Decreased (e.g., −3.15%). |
| | | In some cases, an | In some cases, a |
| | | increase of this | decrease of this |
| | | metabolite of 0.5, 1, | metabolite of 1, 2, 2.5, |
| | | 1.5, 2, or more percent | 3, or more percent as |
| | | as compared to a | compared to a |
| | | reference level can be | reference level can be |
| | | used to identify low | used to identify |
| | | activity disease. | moderate to high |
| | | | activity disease. |
| pyrraline | 263430 | Decreased (e.g., −8.77%). | Increased (e.g., +5.94%). |
| | | In some cases, a | In some cases, an |
| | | decrease of this | increase of this |
| | | metabolite of 1, 2, 3, 4, | metabolite of 1, 2, 3, 4, |
| | | 5, 6, 7, 8, or more | 5, or more percent as |
| | | percent as compared to | compared to a |
| | | a reference level can | reference level can be |
| | | be used to identify low | used to identify |
| | | activity disease. | moderate to high |
| | | | activity disease. |
| cysteinylglycine disulfide | 30506582 | Decreased (e.g., −1.76%). | Increased (e.g., +0.46%). |
| | | In some cases, a | In some cases, an |
| | | decrease of this | increase of this |
| | | metabolite of 0.5, 1, | metabolite of 0.1, 0.2, |
| | | 1.5, 1.6, 1.7, or more | 0.3, 0.4, or more |
| | | percent as compared to | percent as compared to |
| | | a reference level can | a reference level can |
| | | be used to identify low | be used to identify |
| | | activity disease. | moderate to high |
| | | | activity disease. |
| serine | 166907520 | Increased (e.g., +1.20%). | Decreased (e.g., −2.24%). |
| | | In some cases, an | In some cases, a |
| | | increase of this | decrease of this |
| | | metabolite of 0.25, 0.5, | metabolite of 1, 1.5, 2, |
| | | 0.75, 1, or more | 2.1, or more percent as |
| | | percent as compared to | compared to a |
| | | a reference level can | reference level can be |
| | | be used to identify low | used to identify |
| | | activity disease. | moderate to high |
| | | | activity disease. |
| N-acetyltryptophan | 302349 | Increased (e.g., +7.46%). | Decreased (e.g., −8.01%). |
| | | In some cases, an | In some cases, a |
| | | increase of this | decrease of this |
| | | metabolite of 1, 2, 3, 4, | metabolite of 1, 2, 3, 4, |
| | | 5, 6, 7, or more percent | 5, 6, 7, 8, or more |
| | | as compared to a | percent as compared to |
| | | reference level can be | a reference level can |
| | | used to identify low | be used to identify |
| | | activity disease. | moderate to high |
| | | | activity disease. |
| 6-bromotryptophan | 592802.5 | Increased (e.g., +7.80%). | Decreased (e.g., −10.98%). |
| | | In some cases, an | In some cases, a |
| | | increase of this | decrease of this |
| | | metabolite of 1, 2, 3, 4, | metabolite of 1, 2, 3, 4, |
| | | 5, 6, 7, or more percent | 5, 6, 7, 8, 9, 10, 10.5, |
| | | as compared to a | or more percent as |
| | | reference level can be | compared to a |
| | | used to identify low | reference level can be |
| | | activity disease. | used to identify |
| | | | moderate to high |
| | | | activity disease. |
| 1-carboxyethylisoleucine | 88956.5 | Increased (e.g., +0.75%). | Decreased (e.g., −3.00%). |
| | | In some cases, an | In some cases, a |
| | | increase of this | decrease of this |
| | | metabolite of 0.5, 0.6, | metabolite of 1, 1.5, 2, |
| | | 0.7, or more percent as | 2.5, 2.75, or more |
| | | compared to a | percent as compared to |
| | | reference level can be | a reference level can |
| | | used to identify low | be used to identify |
| | | activity disease. | moderate to high |
| | | | activity disease. |
| erucate (22:1n9) | 3775962 | Decreased (e.g., −0.56%). | Increased (e.g., +8.98%). |
| | | In some cases, a | In some cases, an |
| | | decrease of this | increase of this |
| | | metabolite of 0.2, 0.3, | metabolite of 1, 2, 3, 4, |
| | | 0.4, 0.5, or more | 5, 6, 7, 8, 8.5, or more |
| | | percent as compared to | percent as compared to |
| | | a reference level can | a reference level can |
| | | be used to identify low | be used to identify |
| | | activity disease. | moderate to high |
| | | | activity disease. |
| alpha-ketobutyrate | 3890791 | Increased (e.g., | Decreased (e.g., −1.65%). |
| | | increased by +0.69%); | In some cases, a |
| | | In some cases, an | decrease of this |
| | | increase of this | metabolite of 0.5, 1, |
| | | metabolite of 0.4, 0.5, | 1.5, 1.6, or more |
| | | 0.6, or more percent as | percent as compared to |
| | | compared to a | a reference level can |
| | | reference level can be | be used to identify |
| | | used to identify low | moderate to high |
| | | activity disease. | activity disease. |
| N2-acetyl,N6-methyllysine | 955596.5 | Increased (e.g., +7.14%). | Decreased (e.g., −21.14%). |
| | | In some cases, an | In some cases, a |
| | | increase of this | decrease of this |
| | | metabolite of 1, 2, 3, 4, | metabolite of 5, 10, 15, |
| | | 5, 6, 7, or more percent | 17, 19, 20, 21, or more |
| | | as compared to a | percent as compared to |
| | | reference level can be | a reference level can |
| | | used to identify low | be used to identify |
| | | activity disease. | moderate to high |
| | | | activity disease. |
| trigonelline (N′- | 53886060 | Increased (e.g., +18.94%). | Decreased (e.g., −17.84%). |
| methylnicotinate) | | In some cases, an | In some cases, a |
| | | increase of this | decrease of this |
| | | metabolite of 1, 2, 3, 4, | metabolite of 1, 2, 3, 4, |
| | | 5, 6, 7, 8, 9, 10, 11, 12, | 5, 6, 7, 8, 9, 10, 11, 12, |
| | | 13, 14, 15, 16, 17, 18, | 13, 14, 15, 16, 17, or |
| | | or more percent as | more percent as |
| | | compared to a | compared to a |
| | | reference level can be | reference level can be |
| | | used to identify low | used to identify |
| | | activity disease. | moderate to high |
| | | | activity disease. |
| mannose | 31007281 | Decreased (e.g., −7.42%). | Increased (e.g., +10.29%). |
| | | In some cases, a | In some cases, an |
| | | decrease of this | increase of this |
| | | metabolite of 1, 2, 3, 4, | metabolite of 1, 2, 3, 4, |
| | | 5, 6, 7, or more percent | 5, 6, 7, 8, 9, 10, or |
| | | as compared to a | more percent as |
| | | reference level can be | compared to a |
| | | used to identify low | reference level can be |
| | | activity disease. | used to identify |
| | | | moderate to high |
| | | | activity disease. |
| dimethylguanidino valeric | 619222 | Decreased (e.g., −1.18%). | Increased (e.g., +7.38%). |
| acid (DMGV) | | In some cases, a | In some cases, an |
| | | decrease of this | increase of this |
| | | metabolite of 0.5, 0.75, | metabolite of 1, 2, 3, 4, |
| | | 1, 1.1, or more percent | 5, 6, 7, or more percent |
| | | as compared to a | as compared to a |
| | | reference level can be | reference level can be |
| | | used to identify low | used to identify |
| | | activity disease. | moderate to high |
| | | | activity disease. |
| 3-phenylpropionate | 783339.5 | Increased (e.g., +32.09%). | Decreased (e.g., −23.77%). |
| (hydrocinnamate) | | In some cases, an | In some cases, a |
| | | increase of this | decrease of this |
| | | metabolite of 5, 10, 15, | metabolite of 5, 10, 15, |
| | | 20, 25, 30, 31, 32, or | 20, 21, 22, 23, or more |
| | | more percent as | percent as compared to |
| | | compared to a | a reference level can |
| | | reference level can be | be used to identify |
| | | used to identify low | moderate to high |
| | | activity disease. | activity disease. |
| 1-carboxyethylvaline | 367668 | Decreased (e.g., −0.79%). | Increased (e.g., +7.66%). |
| | | In some cases, a | In some cases, an |
| | | decrease of this | increase of this |
| | | metabolite of 0.5, 0.6, | metabolite of 1, 2, 3, 4, |
| | | 0.7, or more percent as | 5, 6, 7, or more percent |
| | | compared to a | as compared to a |
| | | reference level can be | reference level can be |
| | | used to identify low | used to identify |
| | | activity disease. | moderate to high |
| | | | activity disease. |
| tryptophan | 195492424 | Increased (e.g., +4.66%). | Decreased (e.g., −6.07%). |
| | | In some cases, an | In some cases, a |
| | | increase of this | decrease of this |
| | | metabolite of 1, 2, 3, 4, | metabolite of 1, 2, 3, 4, |
| | | or more percent as | 5, 6, or more percent |
| | | compared to a | as compared to a |
| | | reference level can be | reference level can be |
| | | used to identify low | used to identify |
| | | activity disease. | moderate to high |
| | | | activity disease. |
| N-acetylarginine | 3040031 | Increased (e.g., +9.53%). | Decreased (e.g., −12.90%). |
| | | In some cases, an | In some cases, a |
| | | increase of this | decrease of this |
| | | metabolite of 1, 2, 3, 4, | metabolite of 1, 2, 3, 4, |
| | | 5, 6, 7, 8, 9, or more | 5, 6, 7, 8, 9, 10, 11, 12, |
| | | percent as compared to | or more percent as |
| | | a reference level can | compared to a |
| | | be used to identify low | reference level can be |
| | | activity disease. | used to identify |
| | | | moderate to high |
| | | | activity disease. |
| 1-linoleoyl-GPA (18:2) | 284195 | Increased (e.g., +10.37%). | Decreased (e.g., −13.24%). |
| | | In some cases, an | In some cases, a |
| | | increase of this | decrease of this |
| | | metabolite of 1, 2, 3, 4, | metabolite of 1, 2, 3, 4, |
| | | 5, 6, 7, 8, 9, 10, or | 5, 6, 7, 8, 9, 10, 11, 12, |
| | | more percent as | 13, or more percent as |
| | | compared to a | compared to a |
| | | reference level can be | reference level can be |
| | | used to identify low | used to identify |
| | | activity disease. | moderate to high |
| | | | activity disease. |
| beta-hydroxyisovalerate | 2821895 | Decreased (e.g., −0.88%). | Increased (e.g., +0.66%). |
| | | In some cases, a | In some cases, an |
| | | decrease of this | increase of this |
| | | metabolite of 0.5, 0.6, | metabolite of 0.4, 0.5, |
| | | 0.7, 0.8, or more | 0.6, or more percent as |
| | | percent as compared to | compared to a |
| | | a reference level can | reference level can be |
| | | be used to identify low | used to identify |
| | | activity disease. | moderate to high |
| | | | activity disease. |
| stearoyl ethanolamide | 1323003.5 | Decreased (e.g., −11.03%). | Increased (e.g., +8.38%). |
| | | In some cases, a | In some cases, an |
| | | decrease of this | increase of this |
| | | metabolite of 1, 2, 3, 4, | metabolite of 1, 2, 3, 4, |
| | | 5, 6, 7, 8, 9, 10, 10.5, | 5, 6, 7, 8, or more |
| | | or more percent as | percent as compared to |
| | | compared to a | a reference level can |
| | | reference level can be | be used to identify |
| | | used to identify low | moderate to high |
| | | activity disease. | activity disease. |
| gulonate | 6447782 | Increased (e.g., +3.29%). | Decreased (e.g., −3.36%). |
| | | In some cases, an | In some cases, a |
| | | increase of this | decrease of this |
| | | metabolite of 1, 2, 3, | metabolite of 1, 1.5, 2, |
| | | or more percent as | 2.5, 3, 3.3, or more |
| | | compared to a | percent as compared to |
| | | reference level can be | a reference level can |
| | | used to identify low | be used to identify |
| | | activity disease. | moderate to high |
| | | | activity disease. |
| phenol sulfate | 48825586 | Increased (e.g., +7.63%). | Decreased (e.g., −22.10%). |
| | | In some cases, an | In some cases, a |
| | | increase of this | decrease of this |
| | | metabolite of 1, 2, 3, 4, | metabolite of 5, 10, 15, |
| | | 5, 6, 7, or more percent | 17, 18, 19, 20, 21, 22, |
| | | as compared to a | or more percent as |
| | | reference level can be | compared to a |
| | | used to identify low | reference level can be |
| | | activity disease. | used to identify |
| | | | moderate to high |
| | | | activity disease. |
| trimethylamine N-oxide | 22341643 | Decreased (e.g., −2.16%). | Increased (e.g., +2.37%). |
| | | In some cases, a | In some cases, an |
| | | decrease of this | increase of this |
| | | metabolite of 1, 1.5, 2, | metabolite of 1, 1.5, 2, |
| | | 2.1, or more percent as | 2.2, or more percent as |
| | | compared to a | compared to a |
| | | reference level can be | reference level can be |
| | | used to identify low | used to identify |
| | | activity disease. | moderate to high |
| | | | activity disease. |
| 3-hydroxystearate | 1109475 | Decreased (e.g., −9.94%). | Increased (e.g., +9.00%). |
| | | In some cases, a | In some cases, an |
| | | decrease of this | increase of this |
| | | metabolite of 1, 2, 3, 4, | metabolite of 1, 2, 3, 4, |
| | | 5, 6, 7, 8, 9, or more | 5, 6, 7, 8, 8.5, or more |
| | | percent as compared to | percent as compared to |
| | | a reference level can | a reference level can |
| | | be used to identify low | be used to identify |
| | | activity disease. | moderate to high |
| | | | activity disease. |
| branched chain 14:0 | 573385 | Increased (e.g., +2.19%). | Decreased (e.g., −17.51%). |
| dicarboxylic acid | | In some cases, an | In some cases, a |
| | | increase of this | decrease of this |
| | | metabolite of 1, 1.5, 2, | metabolite of 1, 2, 3, 4, |
| | | 2.1, or more percent as | 5, 6, 7, 8, 9, 10, 11, 12, |
| | | compared to a | 13, 14, 15, 16, 17, or |
| | | reference level can be | more percent as |
| | | used to identify low | compared to a |
| | | activity disease. | reference level can be |
| | | | used to identify |
| | | | moderate to high |
| | | | activity disease. |
| bilirubin | 6670012.5 | Increased (e.g., +6.59%). | Decreased (e.g., −8.73%). |
| | | In some cases, an | In some cases, a |
| | | increase of this | decrease of this |
| | | metabolite of 1, 2, 3, 4, | metabolite of 1, 2, 3, 4, |
| | | 5, 6, or more percent | 5, 6, 7, 8, or more |
| | | as compared to a | percent as compared to |
| | | reference level can be | a reference level can |
| | | used to identify low | be used to identify |
| | | activity disease. | moderate to high |
| | | | activity disease. |
| gluconate | 15844674.5 | Decreased (e.g., −0.60%). | Increased (e.g., +1.48%). |
| | | In some cases, a | In some cases, an |
| | | decrease of this | increase of this |
| | | metabolite of 0.3, 0.4, | metabolite of 0.25, 0.5, |
| | | 0.5, or more percent as | 0.75, 1, 1.25, or more |
| | | compared to a | percent as compared to |
| | | reference level can be | a reference level can |
| | | used to identify low | be used to identify |
| | | activity disease. | moderate to high |
| | | | activity disease. |
| linoleoylcarnitine (C18:3) | 830898 | Increased (e.g., +11.72%). | Decreased (e.g., −14.99%). |
| | | In some cases, an | In some cases, a |
| | | increase of this | decrease of this |
| | | metabolite of 1, 2, 3, 4, | metabolite of 1, 2, 3, 4, |
| | | 5, 6, 7, 8, 9, 10, 11, or | 5, 6, 7, 8, 9, 10, 11, 12, |
| | | more percent as | 13, 14, or more percent |
| | | compared to a | as compared to a |
| | | reference level can be | reference level can be |
| | | used to identify low | used to identify |
| | | activity disease. | moderate to high |
| | | | activity disease. |
| palmitoyl ethanolamide | 2865748.5 | Decreased (e.g., −21.28%). | Increased (e.g., +27.67%). |
| | | In some cases, a | In some cases, an |
| | | decrease of this | increase of this |
| | | metabolite of 5, 10, 15, | metabolite of 5, 10, 15, |
| | | 17, 18, 19, 20, 21, or | 20, 25, 26, 27, or more |
| | | more percent as | percent as compared to |
| | | compared to a | a reference level can |
| | | reference level can be | be used to identify |
| | | used to identify low | moderate to high |
| | | activity disease. | activity disease. |
| glucose | 1831276672 | Decreased (e.g., −1.40%). | Increased (e.g., +2.28%). |
| | | In some cases, a | In some cases, an |
| | | decrease of this | increase of this |
| | | metabolite of 0.5, 0.75, | metabolite of 0.25, 0.5, |
| | | 1, 1.25, or more | 0.75, 1, 1.5, 2, or more |
| | | percent as compared to | percent as compared to |
| | | a reference level can | a reference level can |
| | | be used to identify low | be used to identify |
| | | activity disease. | moderate to high |
| | | | activity disease. |
| bilirubin (E,E) | 7864420 | Increased (e.g., +6.39%). | Decreased (e.g., −10.31%). |
| | | In some cases, an | In some cases, a |
| | | increase of this | decrease of this |
| | | metabolite of 1, 2, 3, 4, | metabolite of 1, 2, 3, 4, |
| | | 5, 6, or more percent | 5, 6, 7, 8, 9, 10, or |
| | | as compared to a | more percent as |
| | | reference level can be | compared to a |
| | | used to identify low | reference level can be |
| | | activity disease. | used to identify |
| | | | moderate to high |
| | | | activity disease. |
| glucuronate | 11457360.5 | Decreased (e.g., −3.64%). | Increased (e.g., +4.97%). |
| | | In some cases, a | In some cases, an |
| | | decrease of this | increase of this |
| | | metabolite of 1, 1.5, 2, | metabolite of 1, 2, 3, 4, |
| | | 2.5, 3, 3.5, or more | 4.5, or more percent as |
| | | percent as compared to | compared to a |
| | | a reference level can | reference level can be |
| | | be used to identify low | used to identify |
| | | activity disease. | moderate to high |
| | | | activity disease. |
| eicosenoylcarnitine (C20:1) | 440691.5 | Increased (e.g., +19.42%). | Decreased (e.g., −7.02%). |
| | | In some cases, an | In some cases, a |
| | | increase of this | decrease of this |
| | | metabolite of 5, 10, 15, | metabolite of 1, 2, 3, 4, |
| | | 16, 17, 18, 19, or more | 5, 6, or more percent |
| | | percent as compared to | as compared to a |
| | | a reference level can | reference level can be |
| | | be used to identify low | used to identify |
| | | activity disease. | moderate to high |
| | | | activity disease. |
| lanthionine | 887041 | Increased (e.g., +15.01%). | Decreased (e.g., −12.40%). |
| | | In some cases, an | In some cases, a |
| | | increase of this | decrease of this |
| | | metabolite of 1, 2, 3, 4, | metabolite of 1, 2, 3, 4, |
| | | 5, 6, 7, 8, 9, 10, 11, 12, | 5, 6, 7, 8, 9, 10, 11, 12, |
| | | 13, 14, or more percent | or more percent as |
| | | as compared to a | compared to a |
| | | reference level can be | reference level can be |
| | | used to identify low | used to identify |
| | | activity disease. | moderate to high |
| | | | activity disease. |
| glycoursodeoxycholate | 1865529 | Increased (e.g., +18.85%). | Decreased (e.g., −20.53%). |
| | | In some cases, an | In some cases, a |
| | | increase of this | decrease of this |
| | | metabolite of 5, 10, 15, | metabolite of 5, 10, 15, |
| | | 16, 17, 18, or more | 16, 17, 18, 19, 20, or |
| | | percent as compared to | more percent as |
| | | a reference level can | compared to a |
| | | be used to identify low | reference level can be |
| | | activity disease. | used to identify |
| | | | moderate to high |
| | | | activity disease. |
| biliverdin | 913540 | Increased (e.g., +25.30%). | Decreased (e.g., −14.36%). |
| | | In some cases, an | In some cases, a |
| | | increase of this | decrease of this |
| | | metabolite of 5, 10, 15, | metabolite of 1, 2, 3, 4, |
| | | 20, 21, 22, 23, 24, 25, | 5, 6, 7, 8, 9, 10, 11, 12, |
| | | or more percent as | 13, 14, or more percent |
| | | compared to a | as compared to a |
| | | reference level can be | reference level can be |
| | | used to identify low | used to identify |
| | | activity disease. | moderate to high |
| | | | activity disease. |
| guanidinoacetate | 903718 | Increased (e.g., +2.40%). | Decreased (e.g., −5.67%). |
| | | In some cases, an | In some cases, a |
| | | increase of this | decrease of this |
| | | metabolite of 0.25, 0.5, | metabolite of 1, 2, 3, 4, |
| | | 0.75, 1, 1.5, 2, or more | 5, or more percent as |
| | | percent as compared to | compared to a |
| | | a reference level can | reference level can be |
| | | be used to identify low | used to identify |
| | | activity disease. | moderate to high |
| | | | activity disease. |
| myo-inositol | 25227704 | Increased (e.g., +1.62%). | Decreased (e.g., −2.17%). |
| | | In some cases, an | In some cases, a |
| | | increase of this | decrease of this |
| | | metabolite of 0.25, 0.5, | metabolite of 1, 1.5, 2, |
| | | 0.75, 1, 1.5, or more | 2.1, or more percent as |
| | | percent as compared to | compared to a |
| | | a reference level can | reference level can be |
| | | be used to identify low | used to identify |
| | | activity disease. | moderate to high |
| | | | activity disease. |

^{α}Abundance values were calculated with 76 plasma samples from patients with low disease activity and 52 plasma samples from patients with moderate-high disease activity. Ultra-high-performance liquid chromatography-tandem mass spectrometry (UPLC-MS/MS) was performed by Metabolon Inc.’s Discovery HD4TM platform.
^{β}Abundance differences are differences as compared to the abundance values.

TABLE B
Relative abundance values (mean ± standard deviation) of 51 metabolites for
human plasma samples (measured by metabolon UPLC-MS/MS). The abundance of each
metabolite was rescaled to set the median value for each metabolite equal to 1.

| | mean | Stdev | Min/Max | Mean | Stdev | Min/Max |
|---|---|---|---|---|---|---|
| Metabolites | (LDA^{α}) | (LDA^{α}) | (LDA^{α}) | (HDA^{β}) | (HDA^{β}) | (HDA^{β}) |
| Glycoursodeoxycholate | 2.448 | 3.185 | 0.028/15.312 | 2.858 | 7.260 | 0.014/50.385 |
| Eicosenoylcarnitine (C20:1) | 1.203 | 0.483 | 0.471/2.861 | 0.931 | 0.317 | 0.417/2.179 |
| Branched chain 14:0 | 1.391 | 1.517 | 0.046/8.492 | 1.026 | 1.108 | 0.046/4.890 |
| dicarboxylic acid | | | | | | |
| Lysine | 1.041 | 0.190 | 0.634/1.744 | 0.960 | 0.168 | 0.641/1.398 |
| 3-methylhistidine | 1.413 | 1.410 | 0.066/6.667 | 1.605 | 1.727 | 0.056/8.792 |
| 1-carboxyethylleucine | 1.074 | 0.567 | 0.36/3.35 | 1.198 | 0.807 | 0.321/4.121 |
| Biliverdin | 1.332 | 0.720 | 0.38/3.707 | 0.952 | 0.375 | 0.269/2.134 |
| Trigonelline (N′- | 1.584 | 1.450 | 0.06/6.48 | 0.947 | 0.887 | 0.034/4.428 |
| methylnicotinate) | | | | | | |
| Bilirubin | 1.189 | 0.565 | 0.179/3.223 | 0.990 | 0.428 | 0.316/2.513 |
| Isoursodeoxycholate | 2.386 | 3.215 | 0.109/18.78 | 3.145 | 6.411 | 0.060/34.368 |
| Glucose | 1.017 | 0.174 | 0.629/1.724 | 1.118 | 0.345 | 0.490/2.475 |
| 1-carboxyethylisoleucine | 1.109 | 0.720 | 0.288/4.394 | 1.281 | 1.093 | 0.288/6.108 |
| 3-phenylpropionate | 1.577 | 1.560 | 0.07/6.914 | 1.029 | 1.016 | 0.070/4.613 |
| (hydrocinnamate) | | | | | | |
| Palmitoyl ethanolamide | 2.518 | 3.611 | 0.311/20.131 | 2.678 | 3.072 | 0.314/13.862 |
| Tryptophan | 1.041 | 0.184 | 0.481/1.45 | 0.938 | 0.230 | 0.450/1.649 |
| Dimethylguanidino valeric acid | 1.124 | 0.933 | 0.072/4.745 | 1.211 | 0.969 | 0.072/6.049 |
| (DMGV) | | | | | | |
| Guanidinoacetate | 1.054 | 0.239 | 0.569/1.776 | 0.942 | 0.260 | 0.382/1.648 |
| Phenol sulfate | 1.422 | 1.116 | 0.243/7.888 | 1.064 | 1.114 | 0.087/7.279 |
| Cysteinylglycine disulfide | 1.010 | 0.168 | 0.618/1.415 | 1.043 | 0.164 | 0.673/1.375 |
| Linoleoylcarnitine (C18:3) | 1.202 | 0.512 | 0.359/3.092 | 0.938 | 0.494 | 0.266/3.570 |
| 1-Linoleoyl-GPA (18:2) | 1.108 | 0.543 | 0.273/2.976 | 1.029 | 0.813 | 0.273/4.772 |
| Hypoxanthine | 0.895 | 0.517 | 0.024/2.665 | 1.132 | 0.538 | 0.092/2.632 |
| Mannose | 1.046 | 0.344 | 0.436/1.984 | 1.189 | 0.476 | 0.553/3.123 |
| Pyrraline | 1.184 | 1.044 | 0.104/5.588 | 1.439 | 1.190 | 0.104/5.691 |
| Ectoine | 1.596 | 1.645 | 0.247/9.196 | 1.747 | 1.786 | 0.276/9.152 |
| Trimethylamine N-oxide | 1.099 | 0.576 | 0.35/4.452 | 1.217 | 0.650 | 0.398/3.350 |
| N2-acetyl,N6-methyllysine | 1.761 | 1.844 | 0.088/9.117 | 0.817 | 0.651 | 0.088/3.070 |
| beta-hydroxyisovalerate | 1.035 | 0.301 | 0.37/1.757 | 1.043 | 0.382 | 0.302/2.768 |
| N-acetylarginine | 1.076 | 0.393 | 0.266/2.005 | 0.860 | 0.357 | 0.150/1.917 |
| Stearoyl ethanolamide | 2.267 | 3.130 | 0.487/18.608 | 2.119 | 2.203 | 0.518/11.041 |
| Glucuronate | 1.062 | 0.445 | 0.527/3.379 | 1.408 | 1.004 | 0.524/6.128 |
| 6-bromotryptophan | 1.121 | 0.362 | 0.265/2.12 | 0.929 | 0.346 | 0.218/1.695 |
| Bilirubin (E,E) | 1.243 | 0.613 | 0.231/3.533 | 0.921 | 0.330 | 0.340/1.931 |
| N-acetyltyrosine | 1.185 | 0.460 | 0.435/2.723 | 0.924 | 0.247 | 0.340/1.641 |
| Gluconate | 1.050 | 0.459 | 0.293/3.509 | 1.187 | 0.560 | 0.279/3.298 |
| 1-methylhistidine | 1.091 | 0.406 | 0.462/2.237 | 1.090 | 0.622 | 0.323/4.463 |
| 1,6-anhydroglucose | 1.189 | 1.623 | 0.15/9.931 | 1.676 | 2.754 | 0.150/15.255 |
| (14 or 15)-methylpalmitate | 1.077 | 0.708 | 0.228/5.194 | 1.395 | 1.340 | 0.223/9.042 |
| (a17:0 or i17:0) | | | | | | |
| 4-guanidinobutanoate | 1.130 | 0.635 | 0.324/4.371 | 1.099 | 0.819 | 0.277/5.410 |
| N-acetylneuraminate | 1.017 | 0.268 | 0.541/1.819 | 1.261 | 0.978 | 0.45/6.842 |
| Dihomo-linoleoylcarnitine | 1.150 | 0.400 | 0.381/2.578 | 0.915 | 0.301 | 0.359/1.924 |
| (C20:2) | | | | | | |
| Erucate (22:ln9) | 1.034 | 0.480 | 0.36/3.502 | 1.147 | 0.667 | 0.207/3.106 |
| 1-carboxyethylvaline | 1.067 | 0.552 | 0.342/3.308 | 1.312 | 0.973 | 0.263/5.733 |
| Serine | 1.029 | 0.188 | 0.621/1.571 | 0.968 | 0.177 | 0.502/1.361 |
| Lanthionine | 1.358 | 1.014 | 0.055/5.311 | 1.021 | 0.776 | 0.055/4.540 |
| alpha-ketobutyrate | 1.115 | 0.577 | 0.253/3.498 | 1.248 | 0.932 | 0.275/5.625 |
| Myo-inositol | 1.040 | 0.264 | 0.469/1.801 | 1.047 | 0.328 | 0.588/1.946 |
| N-acetyltryptophan | 1.142 | 0.357 | 0.578/2.824 | 0.928 | 0.271 | 0.390/1.807 |
| Gulonate | 1.087 | 0.422 | 0.447/2.751 | 1.123 | 0.563 | 0.447/2.675 |
| Linoleoylcarnitine (C18:2) | 1.123 | 0.330 | 0.491/1.984 | 0.948 | 0.274 | 0.531/2.029 |
| 3-hydroxystearate | 0.990 | 0.525 | 0.42/4.298 | 1.251 | 0.921 | 0.420/6.967 |

^{α}LDA: Low disease activity: DAS28-CRP > 3.2;
^{β}Moderate-to-high disease activity: DAS28-CRP ≤ 3.2.

A sample (e.g., a plasma sample) obtained from a mammal (e.g., a human) can be assessed for the presence, absence, or level of any appropriate metabolite. A metabolite can be a metabolite that is associated with arthritis (e.g., rheumatoid arthritis). Examples of metabolites (e.g., circulating metabolites) whose presence, absence, or level can be assessed in a sample (e.g., a plasma sample) obtained from a mammal (e.g., a human) as described herein include, without limitation, dihomo-linoleoylcarnitine (C20:2), 6-bromotryptophan, N-acetyltyrosine, eicosenoylcarnitine (C20:1), N-acetylglutamine, bilirubin (E,E), N-acetyltryptophan, N2-acetyl,N6-methyllysine, methionine, biliverdin, hypoxanthine, linoleoylcarnitine (C18:2), glycerophosphorylcholine (GPC), trigonelline (N′-methylnicotinate), tryptophan, linoleoylcarnitine (C18:3), gamma-glutamylmethionine, stearoylcarnitine (C18), N-acetylarginine, 10-undecenoate (11:1n1), N-acetylasparagine, 3-hydroxydecanoylcarnitine, palmitoylcarnitine (C16), glucuronate, glycerophosphoethanolamine, serine, retinal, N-acetyl-2-aminooctanoate, N2,N5-diacetylornithine, carnitine, lysine, N-acetylcitrulline, 3-amino-2-piperidone, 3-hydroxystearate, phenol sulfate, trimethylamine N-oxide, dimethylguanidino valeric acid (DMGV), glycoursodeoxycholate, N-acetylneuraminate, branched chain 14:0 dicarboxylic acid, 1-carboxyethylvaline, (14 or 15)-methylpalmitate (a17:0 or i17:0), isoursodeoxycholate, glucose, 1-methylhistidine, palmitoyl ethanolamide, 3-methylhistidine, 4-guanidinobutanoate, 1-carboxyethylisoleucine, cysteinylglycine disulfide, guanidinoacetate, 1,6-anhydroglucose, pyrraline, mannose, ectoine, 1-linoleoyl-GPA (18:2), erucate (22:1n9), stearoyl ethanolamide, 3-phenylpropionate (hydrocinnamate), beta-hydroxyisovalerate, myo-inositol, gulonate, gluconate, 1-carboxyethylleucine, alpha-ketobutyrate, lanthionine, nonanoylcarnitine (C9), 3-decenoylcarnitine, taurolithocholate 3-sulfate, pyridoxate, 5,6-dihydrouridine, inosine, 2′-O-methyluridine, aconitate [cis or trans], 2-hydroxyphytanate, N-alpha-acetylornithine, creatine, 5-hydroxylysine, N-acetyl-isoputreanine, alpha-ketoglutarate, 1-stearoyl-2-arachidonoyl-GPS (18:0/20:4), hexadecadienoate (16:2n6), S-adenosylhomocysteine (SAH), citraconate/glutaconate, dodecadienoate (12:2), catechol sulfate, octadecanedioylcarnitine (C18-DC), 3-hydroxyadipate, ethylmalonate, 11beta-hydroxyandrosterone glucuronide, bilirubin, isoursodeoxycholate, and glucose.

Any appropriate method can be used to determine the presence, absence, or level of or more (e.g., 50 or 51) metabolites (e.g., circulating metabolites) in a sample (e.g., a plasma sample) obtained from a mammal (e.g., a human). In some cases, the presence, absence, or level of 15 or more (e.g., 50 or 51) metabolites (e.g., circulating metabolites) can be identified as described in Example 1.

In some cases, the methods and materials provided herein can include determining the presence, absence, or level of dihomo-linoleoylcarnitine (C20:2), 6-bromotryptophan, N-acetyltyrosine, eicosenoylcarnitine (C20:1), N-acetylglutamine, bilirubin (E,E), N-acetyltryptophan, N2-acetyl,N6-methyllysine, methionine, biliverdin, hypoxanthine, linoleoylcarnitine (C18:2), glycerophosphorylcholine (GPC), trigonelline (N′-methylnicotinate), tryptophan, linoleoylcarnitine (C18:3), gamma-glutamylmethionine, stearoylcarnitine (C18), N-acetylarginine, 10-undecenoate (11:1n1), N-acetylasparagine, 3-hydroxydecanoylcarnitine, palmitoylcarnitine (C16), glucuronate, glycerophosphoethanolamine, serine, retinal, N-acetyl-2-aminooctanoate, N2,N5-diacetylornithine, carnitine, lysine, N-acetylcitrulline, and 3-amino-2-piperidone.

In some cases, the methods and materials provided herein can include determining the presence, absence, or level of 3-hydroxystearate, phenol sulfate, trimethylamine N-oxide, bilirubin (E,E), serine, dimethylguanidino valeric acid (DMGV), N-acetyltryptophan, glycoursodeoxycholate, N-acetylneuraminate, dihomo-linoleoylcarnitine (C20:2), N-acetyltyrosine, branched chain 14:0 dicarboxylic acid, 1-carboxyethylvaline, (14 or 15)-methylpalmitate (a17:0 or i17:0), isoursodeoxycholate, glucuronate, glucose, linoleoylcarnitine (C18:3), 1-methylhistidine, trigonelline (N′-methylnicotinate), palmitoyl ethanolamide, hypoxanthine, biliverdin, linoleoylcarnitine (C18:2), 3-methylhistidine, N-acetylarginine, 4-guanidinobutanoate, 1-carboxyethylisoleucine, cysteinylglycine disulfide, guanidinoacetate, N2-acetyl,N6-methyllysine, lysine, 1,6-anhydroglucose, pyrraline, mannose, ectoine, 6-bromotryptophan, 1-linoleoyl-GPA (18:2), eicosenoylcarnitine (C20:1), erucate (22:1n9), bilirubin, stearoyl ethanolamide, 3-phenylpropionate (hydrocinnamate), beta-hydroxyisovalerate, myo-inositol, gulonate, gluconate, tryptophan, 1-carboxyethylleucine, alpha-ketobutyrate, and lanthionine.

In some cases, the methods and materials provided herein can include determining the presence, absence, or level of nonanoylcarnitine (C9), 3-decenoylcarnitine, taurolithocholate 3-sulfate, pyridoxate, 5,6-dihydrouridine, inosine, 2′-O-methyluridine, 3-amino-2-piperidone, aconitate [cis or trans], 2-hydroxyphytanate, and N-alpha-acetylornithine.

In some cases, the methods and materials provided herein can include determining the presence, absence, or level of creatine, 5-hydroxylysine, inosine, N-acetyl-isoputreanine, alpha-ketoglutarate, 1-stearoyl-2-arachidonoyl-GPS (18:0/20:4), hexadecadienoate (16:2n6), S-adenosylhomocysteine (SAH), nonanoylcarnitine (C9), citraconate/glutaconate, dodecadienoate (12:2), catechol sulfate, octadecanedioylcarnitine (C18-DC), 3-hydroxyadipate, ethylmalonate, 11beta-hydroxyandrosterone glucuronide, erucate (22:1n9), gamma-glutamylmethionine, and 2-hydroxyphytanate.

In some cases, the methods and materials provided herein can include determining the presence, absence, or level of 6-bromotryptophan, bilirubin (E,E), biliverdin, glucuronate, N-acetyltryptophan, N-acetyltyrosine, serine, and trigonelline (N′-methylnicotinate).

In some cases, the presence, absence, or level of 15 or more (e.g., 50 or 51) metabolites (e.g., circulating metabolites) listed in Table A in a sample (e.g., a plasma sample) obtained from a mammal (e.g., a human) having arthritis (e.g., RA) can be used to determine that the mammal has low disease activity of the arthritis. For example, a mammal (e.g., a human) having arthritis (e.g., RA) that is determined to have an increased level of two or more (e.g., 5, 10, 15, 20, 25, or more) of the following metabolites can be identified as having low disease activity: isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and/or myo-inositol. In some cases, a mammal (e.g., a human) having arthritis (e.g., RA) that is determined to have an increased level of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol can be identified as having low disease activity.

In another example, a mammal (e.g., a human) having arthritis (e.g., RA) that is determined to have a decreased level of two or more (e.g., 5, 10, 15, or more) of the following metabolites can be identified as having low disease activity: (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and/or glucoronate. In some cases, a mammal (e.g., a human) having arthritis (e.g., RA) that is determined to have a decreased level of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate can be identified as having low disease activity.

In some cases, a mammal (e.g., a human) having arthritis (e.g., RA) that is determined to have (a) an increased level of two or more (e.g., 5, 10, 15, 20, 25, or more) of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and/or myo-inositol and (b) a decreased level of two or more (e.g., 5, 10, 15, or more) of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and/or glucoronate can be identified as having low disease activity.

In some cases, a mammal (e.g., a human) having arthritis (e.g., RA) that is determined to have (a) an increased level of two or more (e.g., 5, 10, 15, 20, 25, or more) of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol and (b) a decreased level of two or more (e.g., 5, 10, 15, or more) of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate can be identified as having low disease activity.

In some cases, the presence, absence, or level of 15 or more (e.g., 50 or 51) metabolites (e.g., circulating metabolites) listed in Table A in a sample (e.g., a plasma sample) obtained from a mammal (e.g., a human) having arthritis (e.g., RA) can be used to determine that the mammal has high-to-moderate disease activity of the arthritis. For example, a mammal (e.g., a human) having arthritis (e.g., RA) that is determined to have a decreased level of two or more (e.g., 5, 10, 15, 20, 25, or more) of the following metabolites can be identified as having high-to-moderate disease activity: isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and/or myo-inositol. In some cases, a mammal (e.g., a human) having arthritis (e.g., RA) that is determined to have a decreased level of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol can be identified as having high-to-moderate disease activity.

In another example, a mammal (e.g., a human) having arthritis (e.g., RA) that is determined to have an increased level of two or more (e.g., 5, 10, 15, or more) of the following metabolites can be identified as having high-to-moderate disease activity: (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and/or glucoronate. In some cases, a mammal (e.g., a human) having arthritis (e.g., RA) that is determined to have an increased level of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate can be identified as having high-to-moderate disease activity.

In some cases, a mammal (e.g., a human) having arthritis (e.g., RA) that is determined to have (a) a decreased level of two or more (e.g., 5, 10, 15, 20, 25, or more) of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and/or myo-inositol and (b) an increased level of two or more (e.g., 5, 10, 15, or more) of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and/or glucoronate can be identified as having high-to-moderate disease activity.

In some cases, a mammal (e.g., a human) having arthritis (e.g., RA) that is determined to have (a) a decreased level of two or more (e.g., 5, 10, 15, 20, 25, or more) of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol and (b) an increased level of two or more (e.g., 5, 10, 15, or more) of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate can be identified as having high-to-moderate disease activity.

In some cases, a disease activity of a mammal (e.g., a human) having arthritis (e.g., RA) identified as described herein (e.g., based, at least in part, on the presence, absence, or level of 15 or more metabolites in a blood sample (e.g., a plasma sample) obtained from the mammal) can be confirmed using one or more other arthritis disease activity assessment methods. Examples of arthritis disease activity assessment methods that can be used in combination with the methods and materials described herein include, without limitation, DAS28-CRP, clinical disease activity index (CDAI), and simple disease activity index (SDAI).

This document also provides methods for treating a mammal (e.g., a human) having arthritis (e.g., RA). In some cases, a mammal (e.g., a human) having arthritis (e.g., RA) and assessed as described herein (e.g., for the presence, absence, or level of 15 or more metabolites a blood sample (e.g., a plasma sample) obtained from the mammal) can be administered or instructed to self-administer one or more (e.g., one, two, three, four, five, or more) arthritis treatments, where the one or more arthritis treatments are effective to treat the arthritis within the mammal. For example, a mammal (e.g., a human) having arthritis (e.g., RA) can be administered or instructed to self-administer one or more arthritis treatments selected based, at least in part, on the presence, absence, or level of 15 or more (e.g., 50 or 51) metabolites (e.g., circulating metabolites) assessed in a blood sample (e.g., a plasma sample) obtained from the mammal.

When treating a mammal (e.g., a human) having arthritis (e.g., RA) as described herein (e.g., where the treatment is selected based, at least in part, on the presence, absence, or level of 15 or more (e.g., 50 or 51) metabolites in a blood sample (e.g., a plasma sample) obtained from the mammal), the treatment can be effective to reduce or eliminate one of more symptoms of the arthritis. Examples of symptoms of arthritis include, without limitation, pain, stiffness, tenderness, swelling, redness, decreased range of motion, fatigue, fevers, and weight loss. For example, the methods and materials described herein can be used to reduce one or more symptoms within a mammal having arthritis by, for example, 10, 20, 30, 40, 50, 60, 70, 80, 90, 95, or more percent.

In some cases, a treatment for arthritis (e.g., RA) can include any appropriate arthritis treatment. In some cases, an arthritis treatment can include administering one or more arthritis drugs to a mammal in need thereof. In some cases, an arthritis drug can be a painkiller. In some cases, an arthritis drug can be an opioid. In some cases, an arthritis drug can be a nonsteroidal anti-inflammatory drug (NSAID). In some cases, an arthritis drug can be a disease-modifying antirheumatic drug (DMARD; e.g., conventional synthetic DMARDs (csDMARDs) and biologic disease-modifying antirheumatic drugs (bDMARDs) such as tumor necrosis factor inhibitors (TNFi) bDMARDs and non-TNFi bDMARDs). In some cases, an arthritis drug can be a corticosteroid. Examples of arthritis drugs that can be administered to a mammal having arthritis (e.g., RA) can include, without limitation, acetaminophen, tramadol, oxycodone, hydrocodone, ibuprofen, naproxen, adalimumab, certolizumab, etanercept, infliximab, abatacept, rituximab, tocilizumab, azathioprine, hydroxychloroquine, leflunomide, sulfasalazine, methotrexate, prednisone, cortisone, sarilumab, anakinra, tofacitinib, upadacitinib, baricitinib, methylprednisolone, and combinations thereof. In some cases, an arthritis treatment can include therapy and/or surgery. Examples of therapies and surgeries that can be performed on a mammal having arthritis (e.g., RA) to treat the mammal include, without limitation, physical therapy and surgery (e.g., joint repair surgery, joint replacement surgery, and joint fusion surgery).

When treating a mammal (e.g., a human) having arthritis (e.g., RA) and identified as having low disease activity as described herein (e.g., based, at least in part, on the presence, absence, or level of 15 or more (e.g., 50 or 51) metabolites in a blood sample (e.g., a plasma sample) obtained from the mammal), the mammal can be administered or instructed to self-administer one or more (e.g., one, two, three, four, five, or more) arthritis treatments that are less aggressive and/or less invasive. For example, a mammal (e.g., a human) identified as having low disease activity as described herein may continue on the same drug regimen and/or can be administered one or more arthritis drugs (e.g., adalimumab, certolizumab, etanercept, infliximab, abatacept, rituximab, tocilizumab, sarilumab, azathioprine, hydroxychloroquine, leflunomide, sulfasalazine, methotrexate, prednisone, methylprednisolone, tofacitinib, upadacitinib, baricitinib, and combinations thereof). In some cases, the mammal can continue their prior treatment with one or more of the drugs listed above.

When treating a mammal (e.g., a human) having arthritis (e.g., RA) and identified as having moderate-to-high activity as described herein (e.g., based, at least in part, on the presence, absence, or level of 15 or more (e.g., 50 or 51) metabolites in a blood sample (e.g., a plasma sample) obtained from the mammal), the mammal can be administered or instructed to self-administer one or more (e.g., one, two, three, four, five, or more) arthritis treatments that are more aggressive. For example, a mammal (e.g., a human) identified as having moderate-to-high disease activity as described herein (e.g., based, at least in part, on the presence, absence, or level of 15 or more (e.g., 50 or 51) metabolites in a blood sample (e.g., a plasma sample) obtained from the mammal) despite treatment with methotrexate (or other oral conventional synthetic disease-modifying antirheumatic drug) can be administered one or more biological or targeted synthetic disease-modifying antirheumatic drug (e.g., adalimumab, certolizumab, etanercept, infliximab, abatacept, rituximab, tocilizumab, azathioprine, hydroxychloroquine, leflunomide, sulfasalazine, methotrexate, prednisone, cortisone, methylprednisolone, tofacitinib, upadacitinib, baricitinib, and combinations thereof). If a mammal with arthritis (e.g., RA) has moderate-to-high disease activity based on methods described herein despite treatment with a biologic or targeted synthetic disease-modifying antirheumatic drug (DMARD), for example, then the mammal may be switched to an alternative drug in the same or different class based on mechanism of action (e.g., switch from adalimumab to upadacitinib). If drugs are not appropriate, or if preferred based on other characteristics or preferences, the mammal may undergo surgery (e.g., joint repair surgery, joint replacement surgery, and joint fusion surgery).

In some cases, a mammal (e.g., a human) having arthritis (e.g., RA) and identified as having low activity as described herein can be administered one or more of methotrexate, hydroxychloroquine, sulfasalazine, and leflunomide, while a mammal (e.g., a human) having arthritis (e.g., RA) and identified as having moderate-to-high activity as described herein can be administered one or more of adalimumab, certolizumab, etanercept, golimumab, infliximab, abatacept, tocilizumab, sarilumab, rituximab, tofacitinib, baricitinib, and upadacitinib.

The invention will be further described in the following examples, which do not limit the scope of the invention described in the claims.

### EXAMPLES

### Example 1: Plasma Metabolomic Profiling in Patients with Rheumatoid Arthritis Identifies Biochemical Features Predictive of Quantitative Disease Activity

This Example describes the stratification of RA patients of ‘higher’ and ‘lower’ disease activity groups based on their metabolite signatures.

### Materials and Methods

### Study Population, Subject Enrollment, Sample Collection, and Demographic Characteristics

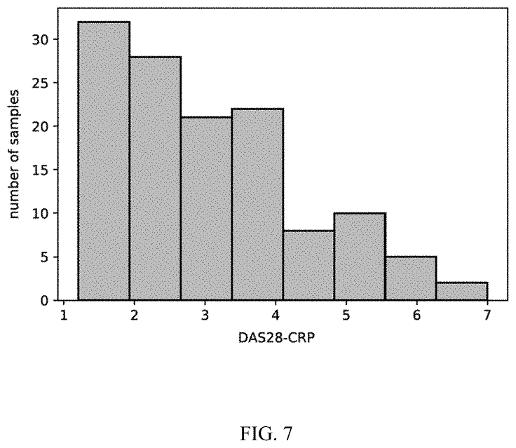

The study population consisted of consecutive patients with RA. Eligibility required patients to be adults 18 years of age or older with a clinical diagnosis of RA by a rheumatologist, fulfilling the American College of Rheumatology/European League Against Rheumatism 2010 revised classification criteria for RA (Aletaha et al., Arthritis & Rheumatism, 62(9):2569-2581 (2010)). A total of 76 patients fulfilled the eligibility criteria, who were partitioned into two groups (Table 1): for the discovery cohort of this study, 64 patients with available blood samples from at least two outpatient visits 6-12 months apart were included (128 total samples); for the validation cohort, 12 patients whose blood samples were available from only a single outpatient visit were included (12 total samples). Demographic and clinical data, including the numbers of tender and swollen joints, patient and evaluator global assessments, CRP (mg/L), body mass index (BMI, kg/m²), smoking status, and results for rheumatoid factor (RF, IU/mL) and anti-cyclic citrullinated peptide antibodies (anti-CCP), were collected from the electronic medical records. The patient samples (140 in total) in the study had established disease with mean age 63.54 (range: 32-86), and 69.7% (53 of 76) were female. Disease activity varied from remission to high disease activity, with a DAS28-CRP mean of 3.0 (range: 1.2-7.0). See FIG. 7 for distribution of DAS28-CRPs corresponding to all study participants.

TABLE 1
Demographic characteristics of study participants.

| | Discovery Cohort^{α} | | Validation Cohort^{β} |
|---|---|---|---|
| | Number of RA patients/samples | | |
| | 64/128 | | 12/12 |
| | Sex of RA patients (female/male) | | |
| | 44/20 | | 9/3 |
| | Visit 1 | Visit 2 | — |
| DAS28-CRP | | | |
| Mean ± SD | 3.1 ± 1.3 | 3.0 ± 1.4 | 2.4 ± 1.3 |
| Range (min-max) | 1.5-7.0 | 1.2-6.6 | 1.7-5.9 |
| Age (years) | | | |
| Mean ± SD | 62.7 ± 10.5 | 63.5 ± 10.6 | 67.8 ± 10.6 |
| Range (min-max) | 32-85 | 33-86 | 54-84 |
| BMI | | | |
| Mean ± SD | 30.6 ± 5.7 | 31.1 ± 6.2 | 27.0 ± 4.1 |
| Range (min-max) | 22.4-45.3 | 22.8-47.8 | 19.0-33.3 |
| N/A (n) | 6 | 6 | 2 |
| Smoking History (n) | | | |
| Current (active within 3 months) | 7 | 5 | 1 |
| Former | 31 | 32 | 3 |
| Never | 25 | 27 | 7 |
| N/A | 1 | 0 | 1 |
| CRP (mg/L) | | | |
| Mean ± SD | 8.91 ± 16.8 | 8.0 ± 12.7 | 11.5 ± 21.7 |
| Range (min-max) | 0.29-113.0 | 0.7-84.0 | 1.0-77.1 |
| RF^{γ} (n) | | | |
| Positive | 36 | — | 6 |
| Negative | 15 | — | 2 |
| N/A | 13 | — | 4 |
| Anti-CCP^{γ} (n) | | | |
| Positive | 44 | — | 5 |
| Negative | 13 | — | 1 |
| N/A | 7 | — | 6 |
| Treatment | | | |
| Methotrexate use (n, %) | 48 (75.0%) | 49 (76.6%) | 7 (58.3%) |
| Methotrexate dose (mg/week) | | | |
| median | 20.0 | 20.0 | 22.5 |
| IQR [Q₁, Q₃] | [15.0, 25.0] | [15.0, 25.0] | [17.5, 25.0] |
| Prednisone use (n, %) | 29 (45.3%) | 28 (43.8%) | 4 (33.3%) |
| Prednisone dose (mg/day) | | | |
| median | 5.0 | 5.0 | 5.0 |
| IQR | [5.0, 7.0] | [5.0, 5.0] | [5.0, 5.0] |
| TNFi-bDMARDs^{δ} (n, %) | 23 (35.9%) | 21 (32.8%) | 3 (25.0%) |
| non-TNFi-bDMARDs^{ε} (n, %) | 6 (9.4%) | 7 (10.9%) | 1 (8.3%) |
| non-methotrexate csDMARDs^{λ} (n, %) | 20 (31.2%) | 27 (42.2%) | 1 (8.3%) |

^{α}Training group. Plasma samples were obtained from patients at two different time-points;
^{β}Test group. Plasma samples were obtained from patients at a single time-point;
^{γ}Reported only for the first visit;
^{δ}adalimumab, certolizumab, etanercept, and infliximab;
^{ε}abatacept, rituximab, and tocilizumab;
^{λ}azathioprine, hydroxychloroquine, leflunomide, and sulfasalazine; N/A, Not available; RF, rheumatoid factor; Anti-CCP, anti-cyclic citrullinated peptide antibodies; IQR, inter-quartile range; bDMARDs, biologic disease-modifying anti-rheumatic drugs; csDMARDs, conventional synthetic disease-modifying anti-rheumatic drugs; an expanded table with further information on demographic and clinical characteristics are as described in Hur et al. (Arthritis Research & Therapy 23: 164 (2021)).

### Metabolomic Profiling

Untargeted metabolomic profiling of plasma samples from both discovery and validation cohorts through ultra-high performance liquid chromatography-tandem mass spectrometry (UPLC-MS/MS) was performed by Metabolon Inc. (Durham, N.C., USA)'s Discovery HD4™ platform.

Sample Accessioning: Following receipt, samples were inventoried and immediately stored at −80° C. Each sample received was accessioned into the Metabolon LIMS system and was assigned by the LIMS a unique identifier that was associated with the original source identifier only. This identifier was used to track all sample handling, tasks, results, etc. The samples (and all derived aliquots) were tracked by the LIMS system. All portions of any sample were automatically assigned their own unique identifiers by the LIMS when a new task was created; the relationship of these samples was also tracked. All samples were maintained at −80° C. until processed.

Sample Preparation: Samples were prepared using the automated MicroLab STAR® system from Hamilton Company. Several recovery standards were added prior to the first step in the extraction process for QC purposes. To remove protein, dissociate small molecules bound to protein or trapped in the precipitated protein matrix, and to recover chemically diverse metabolites, proteins were precipitated with methanol under vigorous shaking for 2 minutes (Glen Mills GenoGrinder 2000) followed by centrifugation. The resulting extract was divided into five fractions: two for analysis by two separate reverse phase (RP)/UPLC-MS/MS methods with positive ion mode electrospray ionization (ESI), one for analysis by RP/UPLC-MS/MS with negative ion mode ESI, one for analysis by HILIC/UPLC-MS/MS with negative ion mode ESI, and one sample was reserved for backup. Samples were placed briefly on a TurboVap® (Zymark) to remove the organic solvent. The sample extracts were stored overnight under nitrogen before preparation for analysis.

Ultrahigh Performance Liquid Chromatography-Tandem Mass Spectroscopy (UPLC-MS/MS): All methods utilized a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract was dried then reconstituted in solvents compatible to each of the four methods. Each reconstitution solvent contained a series of standards at fixed concentrations to ensure injection and chromatographic consistency. One aliquot was analyzed using acidic positive ion conditions, chromatographically optimized for more hydrophilic compounds. In this method, the extract was gradient eluted from a C18 column (Waters UPLC BEH C18-2.1×100 mm, 1.7 μm) using water and methanol, containing 0.05% perfluoropentanoic acid (PFPA) and 0.1% formic acid (FA). Another aliquot was also analyzed using acidic positive ion conditions, however it was chromatographically optimized for more hydrophobic compounds. In this method, the extract was gradient eluted from the same afore mentioned C18 column using methanol, acetonitrile, water, 0.05% PFPA and 0.01% FA and was operated at an overall higher organic content. Another aliquot was analyzed using basic negative ion optimized conditions using a separate dedicated C18 column. The basic extracts were gradient eluted from the column using methanol and water, however with 6.5 mM Ammonium Bicarbonate at pH 8. The fourth aliquot was analyzed via negative ionization following elution from a HILIC column (Waters UPLC BEH Amide 2.1×150 mm, 1.7 μm) using a gradient consisting of water and acetonitrile with 10 mM Ammonium Formate, pH 10.8. The MS analysis alternated between MS and data-dependent MSn scans using dynamic exclusion. The scan range varied slighted between methods but covered 70-1000 m/z. Raw data files were archived and extracted as described below. Metabolites were identified by automated comparison of the ion features in the experimental samples to a reference library of chemical standard entries that included retention time, molecular weight (m/z), preferred adducts, and in-source fragments as well as associated MS spectra, and were curated by visual inspection for quality control using software developed at Metabolon.

Data Extraction and Compound Identification: Raw data was extracted, peak-identified and QC processed using Metabolon's hardware and software. These systems are built on a web-service platform utilizing Microsoft's NET technologies, which run on high-performance application servers and fiber-channel storage arrays in clusters to provide active failover and load-balancing. Compounds were identified by comparison to library entries of purified standards or recurrent unknown entities. Metabolon maintains a library based on authenticated standards that contains the retention time/index (RI), mass to charge ratio (m z), and chromatographic data (including MS/MS spectral data) on all molecules present in the library. Furthermore, biochemical identifications are based on three criteria: retention index within a narrow RI window of the proposed identification, accurate mass match to the library +/−10 ppm, and the MS/MS forward and reverse scores between the experimental data and authentic standards. The MS/MS scores are based on a comparison of the ions present in the experimental spectrum to the ions present in the library spectrum. While there may be similarities between these molecules based on one of these factors, the use of all three data points can be utilized to distinguish and differentiate biochemicals. More than 3300 commercially available purified standard compounds have been acquired and registered into LIMS for analysis on all platforms for determination of their analytical characteristics. Additional mass spectral entries have been created for structurally unnamed biochemicals, which have been identified by virtue of their recurrent nature (both chromatographic and mass spectral). These compounds have the potential to be identified by future acquisition of a matching purified standard or by classical structural analysis.

### Analysis Workflow

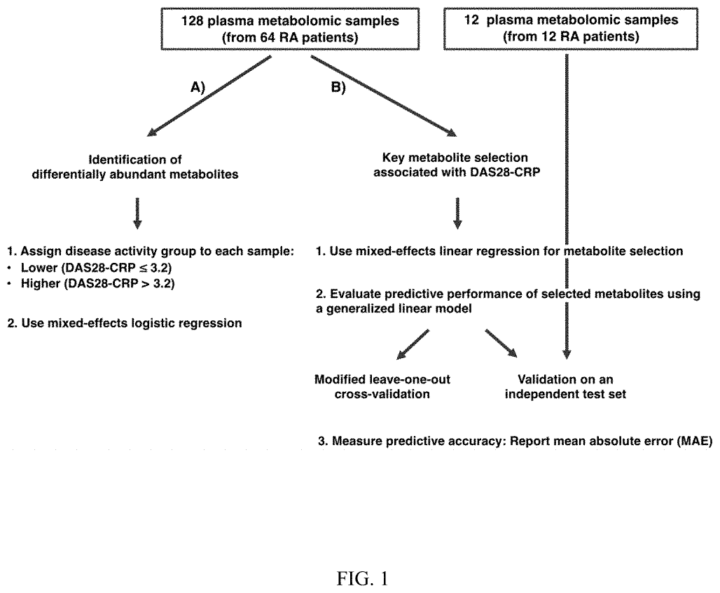

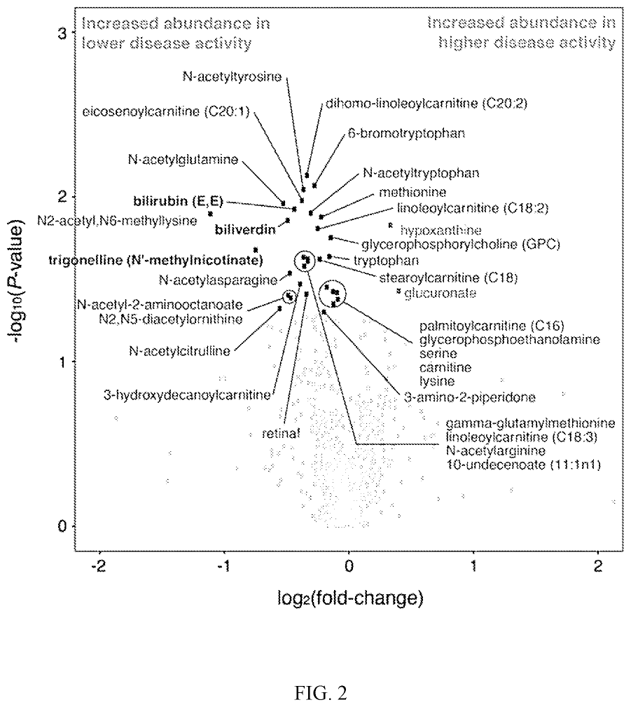

FIG. 1 provides a summary of the analytic strategy used on the 128 plasma samples of the discovery cohort to identify associations between metabolites and RA disease activity. The analysis workflow consists of two complementary approaches: Using mixed-effects logistic regression, the first approach identifies metabolites that are differentially abundant between higher and lower disease activity groups, which were determined by DAS28-CRP scores (FIG. 1A); the second approach uses mixed-effects linear regression to model the relationship between DAS28-CRP and metabolite abundances, allowing the detection of key biochemical features that associate with quantitative disease activity (FIG. 1). To test the predictive accuracy of these selected features when incorporated into a generalized linear model, an additional cohort of twelve plasma metabolomic profiles (from twelve RA patients obtained at single time-points) was collected as an independent validation set.

### Pre-Processing of Metabolomic Profiling Data

Statistical analyses on untargeted metabolomic data were performed using scaled imputed data provided by Metabolon, Inc. Briefly, the raw data were normalized to account for inter-day variation, which is a result of UPLC-MS/MS runs over multiple days, then the peak intensities were rescaled to set each metabolite's median equal to 1. Missing values were then imputed with the minimum observed value of the metabolite across all samples, finally yielding the scaled imputed data. In addition, metabolites with missing values in over 20% of the entire samples were removed, resulting in 686 metabolites remaining for further analysis. R (v3.6.1), lme4 package (v1.1.21), Python3 (v3.7.5), and sklearn (v0.22.2) were used to perform all data pre-processing and statistical analyses.

### Delineation of RA Disease Activity Groups

Samples from RA patients were divided into two disease activity groups based upon DAS28-CRP: ‘lower’ (DAS28-CRP≤3.2, n=76) and ‘higher’ (DAS28-CRP>3.2, n=52). These pre-defined two disease activity groups were used as the nominal response variable in a mixed-effects logistic regression model to identify differentially abundant metabolites between the two groups. The demographic characteristics of samples (n=128) divided into lower and higher disease activity are as described in Hur et al. (Arthritis Research & Therapy 23:164 (2021)).

Identification of Differentially Abundant Metabolites while Controlling for Confounding Factors

The following patient characteristics were examined to identify potential confounding factors in the association between plasma metabolites and disease activity (i.e., higher or lower disease activity): age, sex, BMI, smoking history, and treatment use (for methotrexate, prednisone, non-methotrexate csDMARDs, TNFi-bDMARDs, and non-TNFi-bDMARDS). Based upon the Fisher's exact test, patient age (age ≤60, age >60) and sex (male, female) were observed to have statistically significant associations with the two disease activity groups; the P-value for age and sex was P=0.01 (odds ratio [OR]=2.74, 95% confidence interval [CI]=1.15-6.73) and P=0.02 (OR=0.37, 95% CI=0.14-0.88), respectively. On the other hand, no statistically significant associations were observed between these two disease activity groups and BMI (BMI≤30, BMI>30; P=0.32), disease duration (duration ≤9 years, duration >9 years; P=0.14), smoking history (smoked at least once, never smoked; P=0.36), or treatment use (user, non-user) for methotrexate (P=0.83), prednisone (P=0.58), TNFi-bDMARDs (e.g., adalimumab, certolizumab, etanercept, and infliximab; P=0.18), non-TNFi-bDMARDs (e.g., abatacept, rituximab, and tocilizumab; P=0.76), or other non-methotrexate csDMARDs (e.g., azathioprine, hydroxychloroquine, leflunomide, and sulfasalazine; P=0.71). In addition, no significant changes in treatment use were observed between the two visits; P-values of the associations between treatment use and time-point based upon McNemar's Chi-squared test for paired nominal data were as follows: methotrexate (P=1), prednisone (P=1), TNFi-bDMARDs (P=0.75), non-TNFi-bDMARDs (P=1), and non-methotrexate csDMARDs (P=0.07). Therefore, the mixed-effects logistic regression model was adjusted for age and sex as fixed effects, but not for all other aforementioned covariates. Patient ID was considered as a random effect in the model to account for intra-subject variance due to having repeated measurements from a single patient. By controlling for patient ID (which are unique to each patient) as a random effect, the non-independence in the data are acknowledged. Leveraging multiple samples from the same patient allows us to compensate for the small number of samples in higher disease activity (DAS28-CRP>3.2) in each visit (visit 1 and visit 2 having 25 and 27 samples, respectively) by maximizing the degree of freedom for the quantitative disease activity measure, and thereby to boost statistical power. No significant difference was observed in DAS28-CRP between visit 1 and visit 2 (P=0.98, Wilcoxon signed-rank test). Metabolites whose corresponding coefficients of the regression model were of P-value <0.05 were considered as differentially abundant, that is, having a statistically significant association with disease activity group.

Selection of Metabolites Associated with DAS28-CRP

Selection of metabolites associated with DAS28-CRP was performed with a mixed-effects linear regression model (DAS28-CRP as the continuous response variable), which controls for fixed effects (scaled metabolite abundances, patients' age and sex) and for random effects (patient ID). Satterthwaite's degrees of freedom method supported by lmerTest (v3.1.1) was applied to test for the statistical significance (P-value) of associations between metabolites and DAS28-CRP. P-values were retrieved from the corresponding regression coefficients of the predictor variables.

### Evaluation of Predictive Performance of DAS28-CRP-Associated Metabolites

A generalized linear model (GLM) was used to estimate DAS28-CRP scores using the aforementioned significantly associated metabolites as predictor variables. Predictive performance of the parameterized model was evaluated by two different techniques: First, a modified leave-one-out cross-validation approach was applied to the 128 samples of the training group (discovery cohort). More specifically, in each cross-validation loop, both samples from the same patient were allocated as an internal validation set, while all remaining samples (126 samples from 63 patients) were used to select metabolites significantly associated with DAS28-CRP (P<0.05). These selected biochemical features were then included in a GLM for predicting DAS28-CRP scores of the remaining two samples (of the internal validation group) from their metabolite abundances. The second approach considers testing a GLM, which was composed of the DAS28-CRP-associated metabolites identified from all 128 samples of the training group, on the independent validation group of 12 plasma samples (validation cohort). For both techniques, model performance was reported using mean absolute error (MAE) and standard deviation (SD).

Identification of Metabolites Associated with Treatment Use

A marginal, mixed-effects linear regression model was used to relate metabolite abundance with treatment use. Scaled metabolite abundance, treatment use, and patient ID was set as the response variable, predictor variable (fixed effect), and random effect, respectively. Use of the following treatments was assessed individually: methotrexate, prednisone, non-methotrexate csDMARDs, TNFi-bDMARDs, and non-TNFi-bDMARDs (names of individual drugs in each treatment group are provided in the footnote of Table 1). P-values were retrieved from the corresponding regression coefficient of the predictor variable (i.e., use or non-use), and a significance of P<0.05 was reported as statistically significant.

### Identification of Differentially Abundant Metabolites Between Two CRP Groups

Metabolites that are significantly associated with disease activity groups and DAS28-CRP scores were further investigated to find those associated with patient groups delineated by CRP levels. First, all samples were divided into two groups as follows: ‘high-CRP’ (CRP>3.0 mg/L, n=52) and ‘low-CRP’ (CRP≤3.0 mg/L, n=76). Next, a marginal, mixed-effects linear regression model was used to define the abundance of a metabolite based upon the following fixed effects: CRP group, sex, age, smoking history, and treatment with prednisone, methotrexate, non-methotrexate csDMARDs, TNFi-bDMARDs; or non-TNFi-bDMARDs. Additionally, patient ID was treated as a random effect. Any covariates whose association with metabolite abundance was statistically significant (i.e., P-value of the corresponding regression coefficient <0.05) were then included in an adjusted mixed model for metabolite abundance. Finally, metabolites were considered as differentially abundant between the two CRP groups if the association between metabolite abundance and CRP group was still found to be significant in the adjusted model (P<0.05).

### Results

### Differentially Abundant Metabolites Between Higher and Lower Disease Activity Groups

As shown in our analysis workflow (FIG. 1), we first sought metabolites that were significantly different in abundance between two major disease activity groups. For this, the 128 metabolomic profiles were divided into two major categories (‘higher’ vs. ‘lower’) based upon the reported disease activity of the corresponding patient at the time of sample collection. Using a mixed-effects logistic regression model, 33 metabolites were identified as differentially abundant between higher (n=52) and lower (n=76) DAS28-CRP groups (FIG. 2). Most of these metabolites (31 of 33) were observed to have significantly increased abundances in lower disease activity, whereas the remaining two (glucuronate and hypoxanthine) were found to be significantly increased in higher disease activity. Of the 31 metabolites increased in lower disease activity, seven metabolites (3-hydroxydecanoylcarnitine, dihomo-linoleoylcarnitine (C20:2), eicosenoylcarnitine (C20:1), linoleoylcarnitine (C18:3), linoleoylcarnitine (C18:2), stearoylcarnitine (C18), palmitoylcarnitine (C16)) are a part of acylcarnitine metabolism, and represent a 3.6-fold enrichment in metabolites involved in this particular pathway (P=1.9×10⁻³, hypergeometric test). It is important to note that the differences seen are relatively small in terms of fold-change, with most of the metabolites varying by 1.1-1.3 fold. Despite these subtle differences within RA patients of varying disease activities, statistically significant signal was obtained even after considering and controlling for all known potentially confounding factors (which often leads to reduction in statistical power), while adhering to our cut-offs for statistical significance (P<0.05).

N2-acetyl,N6-methyllysine (|log 2(FC)|=1.11, P=1.26×10⁻²) and trigonelline (N′-methylnicotinate) (|log 2(FC)|=0.74, P=2.09×10⁻²), which were both found to have increased abundance in lower disease activity, were the top two metabolites having the largest fold-changes between the two groups. Biliverdin (|log₂(FC)|=0.48, P=1.38×10⁻²) and bilirubin (E,E) (|log 2(FC)|=0.43, P=1.18×10⁻²), which are known metabolic products of the heme catabolic pathway, were also observed to have significantly increased abundances in lower disease activity. The full list of differentially abundant metabolites and their associated pathways are shown in Table 2.

TABLE 2
Differentially abundant metabolites between lower (DAS28-CRP ≤ 3.2)
and higher (DAS28-CRP > 3.2) disease activity groups.

| | | Mean | Mean | | |
|---|---|---|---|---|---|
| | | (lower | (higher | | |
| | | disease | disease | | |
| Chemical | | activity | activity | log₂ fold | Absolute log₂ |
| ID^{α} | P-Value | group) | group) | change^{β} | (fold change) |
| 100015839 | 0.00740518 | 1.150059211 | 0.915419231 | −0.329203635 | 0.329203635 |
| 100020414 | 0.00853385 | 1.120727632 | 0.929205769 | −0.270365689 | 0.270365689 |
| 100001104 | 0.00904587 | 1.184696053 | 0.923844231 | −0.358795442 | 0.358795442 |
| 100015838 | 0.01050137 | 1.203475 | 0.931028846 | −0.3703084 | 0.3703084 |
| 100001253 | 0.01095929 | 1.364868421 | 0.9508 | −0.521548068 | 0.521548068 |
| 100001950 | 0.01188551 | 1.242778947 | 0.92055 | −0.433001718 | 0.433001718 |
| 100001254 | 0.01254553 | 1.142303947 | 0.928190385 | −0.299453921 | 0.299453921 |
| 100020546 | 0.01267987 | 1.760740789 | 0.817223077 | −1.107380687 | 1.107380687 |
| 415 | 0.01324431 | 1.074835526 | 0.925073077 | −0.21647667 | 0.21647667 |
| 250 | 0.01388466 | 1.33235 | 0.952098077 | −0.484791019 | 0.484791019 |
| 171 | 0.01481688 | 0.894672368 | 1.132363462 | 0.339905738 | 0.339905738 |
| 100003151 | 0.01557361 | 1.123313158 | 0.948419231 | −0.244163358 | 0.244163358 |
| 100000269 | 0.01768727 | 1.088839474 | 0.988378846 | −0.139655236 | 0.139655236 |
| 100001092 | 0.02099958 | 1.584206579 | 0.947273077 | −0.741908187 | 0.741908187 |
| 565 | 0.02305521 | 1.040543421 | 0.938330769 | −0.14916869 | 0.14916869 |
| 100015831 | 0.02323304 | 1.202006579 | 0.937559615 | −0.358462459 | 0.358462459 |
| 100001313 | 0.02380465 | 1.202826316 | 0.960146154 | −0.325102402 | 0.325102402 |
| 100001391 | 0.02389079 | 1.108465789 | 0.944884615 | −0.230354175 | 0.230354175 |
| 100001266 | 0.02463805 | 1.076190789 | 0.860423077 | −0.322815741 | 0.322815741 |
| 100001197 | 0.02633702 | 1.215452632 | 0.952098077 | −0.35231157 | 0.35231157 |
| 100001257 | 0.02908223 | 1.2572 | 0.909661538 | −0.466812417 | 0.466812417 |
| 100021141 | 0.03388509 | 1.260892105 | 0.965390385 | −0.385260467 | 0.385260467 |
| 100000776 | 0.03528276 | 1.095951316 | 0.974332692 | −0.169697333 | 0.169697333 |
| 100000257 | 0.03730717 | 1.062377632 | 1.407644231 | 0.405986076 | 0.405986076 |
| 100001620 | 0.03774306 | 1.066026316 | 0.980257692 | −0.12101009 | 0.12101009 |
| 503 | 0.0381436 | 1.029375 | 0.967703846 | −0.089131149 | 0.089131149 |
| 100004088 | 0.03913854 | 1.114244737 | 0.884019231 | −0.333916487 | 0.333916487 |
| 100020204 | 0.03969724 | 1.404751316 | 1.005826923 | −0.481932676 | 0.481932676 |
| 100004575 | 0.04120342 | 1.23235 | 0.895746154 | −0.460250205 | 0.460250205 |
| 100000007 | 0.04185476 | 1.037297368 | 0.979798077 | −0.082273175 | 0.082273175 |
| 407 | 0.04472243 | 1.041238158 | 0.959955769 | −0.117260249 | 0.117260249 |
| 100001577 | 0.04776566 | 1.364571053 | 0.932911538 | −0.548635325 | 0.548635325 |
| 100020361 | 0.04991135 | 1.041161842 | 0.910038462 | −0.194194919 | 0.194194919 |

| | | Metabolic | |
|---|---|---|---|
| Chemical | | Super- | Metabolic Sub- |
| ID^{α} | Name of Metabolite | pathway^{γ} | pathway^{g} |
| 100015839 | Dihomo-linoleoylcarnitine | Lipid | Fatty Acid Metabolism |
| | (C20:2) | | (Acyl Carnitine, |
| | | | Polyunsaturated) |
| 100020414 | 6-bromotryptophan | Amino Acid | Tryptophan Metabolism |
| 100001104 | N-acetyltyrosine | Amino Acid | Tyrosine Metabolism |
| 100015838 | Eicosenoylcarnitine | Lipid | Fatty Acid Metabolism |
| | (C20:1) | | (Acyl Carnitine, |
| | | | Monounsaturated) |
| 100001253 | N-acetylglutamine | Amino Acid | Glutamate Metabolism |
| 100001950 | Bilirubin (E,E) | Cofactors | Hemoglobin and |
| | | and Vitamins | Porphyrin Metabolism |
| 100001254 | N-acetyltryptophan | Amino Acid | Tryptophan Metabolism |
| 100020546 | N2-acetyl,N6-methyllysine | Amino Acid | Lysine Metabolism |
| 415 | Methionine | Amino Acid | Methionine, Cysteine, |
| | | | SAM and Taurine |
| | | | Metabolism |
| 250 | Biliverdin | Cofactors | Hemoglobin and |
| | | and Vitamins | Porphyrin Metabolism |
| 171 | Hypoxanthine | Nucleotide | Purine Metabolism, |
| | | | (Hypo)Xanthine/Inosine |
| | | | containing |
| 100003151 | Linoleoylcarnitine (C18:2) | Lipid | Fatty Acid Metabolism |
| | | | (Acyl Carnitine, |
| | | | Polyunsaturated) |
| 100000269 | Glycerophosphorylcholine | Lipid | Phospholipid |
| | (GPC) | | Metabolism |
| 100001092 | Trigonelline (N′- | Cofactors | Nicotinate and |
| | methylnicotinate) | and Vitamins | Nicotinamide |
| | | | Metabolism |
| 565 | Tryptophan | Amino Acid | Tryptophan Metabolism |
| 100015831 | Linoleoylcarnitine (C18:3) | Lipid | Fatty Acid Metabolism |
| | | | (Acyl Carnitine, |
| | | | Polyunsaturated) |
| 100001313 | Gamma-glutamylmethionine | Peptide | Gamma-glutamyl Amino |
| | | | Acid |
| 100001391 | Stearoylcarnitine (C18) | Lipid | Fatty Acid Metabolism |
| | | | (Acyl Carnitine, Long |
| | | | Chain Saturated) |
| 100001266 | N-acetylarginine | Amino Acid | Urea cycle; Arginine |
| | | | and Proline Metabolism |
| 100001197 | 10-undecenoate (11:1n1) | Lipid | Medium Chain Fatty |
| | | | Acid |
| 100001257 | N-acetylasparagine | Amino Acid | Alanine and Aspartate |
| | | | Metabolism |
| 100021141 | 3-hydroxydecanoylcarnitine | Lipid | Fatty Acid Metabolism |
| | | | (Acyl Carnitine, |
| | | | Hydroxy) |
| 100000776 | Palmitoylcarnitine (C16) | Lipid | Fatty Acid Metabolism |
| | | | (Acyl Carnitine, Long |
| | | | Chain Saturated) |
| 100000257 | Glucuronate | Carbohydrate | Aminosugar Metabolism |
| 100001620 | Glycerophosphoethanolamine | Lipid | Phospholipid |
| | | | Metabolism |
| 503 | Serine | Amino Acid | Glycine, Serine and |
| | | | Threonine Metabolism |
| 100004088 | Retinal | Cofactors | Vitamin A Metabolism |
| | | and Vitamins | |
| 100020204 | N-acetyl-2-aminooctanoate | Lipid | Fatty Acid, Amino |
| 100004575 | N2,N5-diacetylornithine | Amino Acid | Urea cycle; Arginine |
| | | | and Proline Metabolism |
| 100000007 | Carnitine | Lipid | Carnitine Metabolism |
| 407 | Lysine | Amino Acid | Lysine Metabolism |
| 100001577 | N-acetylcitrulline | Amino Acid | Urea cycle; Arginine |
| | | | and Proline Metabolism |
| 100020361 | 3-amino-2-piperidone | Amino Acid | Urea cycle; Arginine |
| | | | and Proline Metabolism |

^{α}Chemical ID defined by Metabolon’s Discovery HD4 ™ platform
^{β}fold change = mean (higher disease activity group)/mean (lower disease activity group)
^{γ}Super-pathways and sub-pathways were defined by Metabolon's Discovery HD4 ™ platform

### Metabolic Feature Selection Improves DAS28-CRP Prediction Accuracy

Having uncovered metabolites demonstrating altered abundance between two major disease activity groups, it was next investigated whether quantitative disease activity can be predicted with plasma metabolomes. Mixed-effects linear regression models were used to select metabolites significantly associated with DAS28-CRP. Afterwards, the abundances of the selected metabolic features were incorporated into a GLM to predict DAS28-CRP. For comparison purposes, a GLM was constructed without metabolic feature selection, and thereby taking into consideration all features of a metabolomic profile.

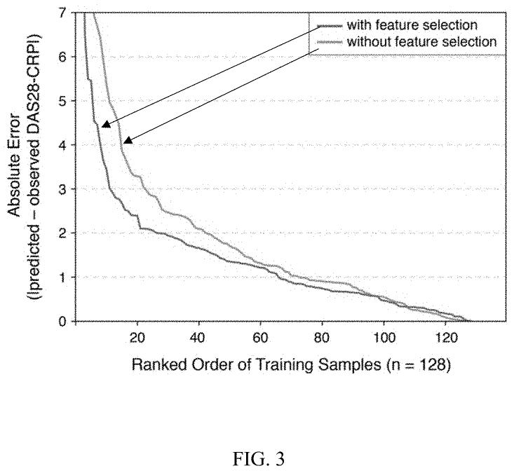

When applying a modified leave-one-out cross-validation technique to the training group samples (n=128), it was found that the GLM incorporating metabolites that were significantly associated with DAS28-CRP outperformed the model without feature selection (i.e., using all metabolites). As shown in FIG. 3, the distribution of absolute errors between the observed and predicted DAS28-CRP scores was smaller (with respect to the cumulative area under the error curve) for the GLM with feature selection than that without feature selection. To this point, the prediction MAE (±SD) of the GLM with and without feature selection was 1.51 (±1.89) and 2.02 (±2.52), respectively.

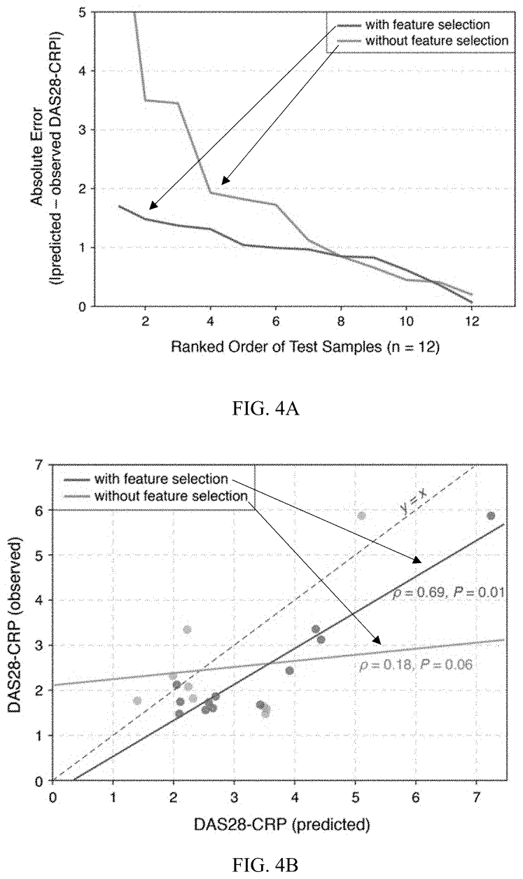

Having confirmed that feature selection can lead to a more accurate prediction model in cross-validation, the same scheme was applied to all metabolome samples of the discovery cohort to obtain a final set of metabolites associated with DAS28-CRP (P<0.05). After adjusting for potential confounding factors, this resulted in a collection of 51 plasma metabolites (Table 3). These metabolites were used to construct a final GLM, whose predictive accuracy was tested on an independent validation cohort (n=12) of plasma metabolomic profiles from twelve RA patients (this additional cohort was not drawn from the same population distribution from which the features were derived). On this previously unseen cohort, the GLM constructed with only the 51 selected metabolites performed considerably better than the model without the feature selection scheme by over two-fold (FIG. 4A); the prediction MAE of the GLM with and without feature selection was 0.97 (+0.47) and 2.01 (+2.18), respectively. Likewise, when the actual and predicted DAS28-CRPs were plotted together for both GLMs (FIG. 4B), it was found that the model with the selection scheme performed more favorably. More specifically, a stronger correlation between the actual and predicted disease activity scores was observed in the model with feature selection (Spearman's ρ=0.69, P=1.40×10⁻², 95% CI: [0.18, 0.90]) compared to the model without (Spearman's ρ=0.18, P=5.72×10⁻², 95% CI: [−0.44, 0.68]).

TABLE 3
Plasma metabolites significantly associated with DAS28-CRP.

| | | | | Regression | |
|---|---|---|---|---|---|
| Metabolite Name | Super-Pathway^{α} | Sub-Pathway^{α} | HMDB ID^{β} | Coefficient^{γ} | P-value^{δ} |
| 3-hydroxystearate | Lipid | Fatty Acid, Monohydroxy | N/A | 0.418 | 0.002 |
| Phenol sulfate | Amino Acid | Tyrosine Metabolism | HMDB60015 | −0.265 | 0.003 |
| Trimethylamine N-oxide | Lipid | Phospholipid Metabolism | HMDB00925 | 0.485 | 0.004 |
| Bilirubin (E,E) | Cofactors and | Hemoglobin and Porphyrin Metabolism | N/A | −0.612 | 0.007 |
| | Vitamins | | | | |
| Serine | Amino Acid | Glycine, Serine and Threonine | HMDB00187 | −1.594 | 0.010 |
| | | Metabolism | | | |
| Dimethylguanidino valeric acid (DMGV) | Amino Acid | Urea cycle; Arginine and Proline | N/A | 0.325 | 0.011 |
| | | Metabolism | | | |
| N-acetyltryptophan | Amino Acid | Tryptophan Metabolism | HMDB13713 | −0.918 | 0.012 |
| Glycoursodeoxycholate | Lipid | Secondary Bile Acid Metabolism | HMDB00708 | 0.051 | 0.012 |
| N-acetylneuraminate | Carbohydrate | Aminosugar Metabolism | HMDB00230 | 0.470 | 0.013 |
| Dihomo-linoleoylcarnitine (C20:2) | Lipid | Fatty Acid Metabolism (Acyl Carnitine, | N/A | −0.745 | 0.013 |
| | | Polyunsaturated) | | | |
| N-acetyltyrosine | Amino Acid | Tyrosine Metabolism | HMDB00866 | −0.713 | 0.014 |
| Branched chain 14:0 dicarboxylic acid | Lipid | Fatty Acid, Dicarboxylate | N/A | −0.201 | 0.014 |
| 1-carboxyethylvaline | Amino Acid | Leucine, Isoleucine and Valine | N/A | 0.408 | 0.015 |
| | | Metabolism | | | |
| (14 or 15)-methylpalmitate | Lipid | Fatty Acid, Branched | N/A | 0.227 | 0.017 |
| (a17:0 or i17:0) | | | | | |
| Isoursodeoxycholate | Lipid | Secondary Bile Acid Metabolism | HMDB00686 | 0.059 | 0.018 |
| Glucuronate | Carbohydrate | Aminosugar Metabolism | HMDB00127 | 0.396 | 0.019 |
| Glucose | Carbohydrate | Glycolysis, Gluconeogenesis, and | HMDB00122 | 1.107 | 0.019 |
| | | Pyruvate Metabolism | | | |
| Linoleoylcarnitine (C18:3) | Lipid | Fatty Acid Metabolism (Acyl Carnitine, | N/A | −0.534 | 0.020 |
| | | Polyunsaturated) | | | |
| 1-methylhistidine | Amino Acid | Histidine Metabolism | HMDB00001 | 0.580 | 0.020 |
| Trigonelline (N′-methylnicotinate) | Cofactors and | Nicotinate and Nicotinamide Metabolism | HMDB00875 | −0.227 | 0.020 |
| | Vitamins | | | | |
| Palmitoyl ethanolamide | Lipid | Endocannabinoid | HMDB02100 | 0.067 | 0.020 |
| Hypoxanthine | Nucleotide | Purine Metabolism, | HMDB00157 | 0.482 | 0.022 |
| | | (Hypo)Xanthine/Inosine containing | | | |
| Biliverdin | Cofactors and | Hemoglobin and Porphyrin Metabolism | HMDB01008 | −0.436 | 0.022 |
| | Vitamins | | | | |
| Linoleoylcarnitine (C18:2) | Lipid | Fatty Acid Metabolism (Acyl Carnitine, | HMDB06469 | −0.814 | 0.023 |
| | | Polyunsaturated) | | | |
| 3-methylhistidine | Amino Acid | Histidine Metabolism | HMDB00479 | 0.140 | 0.025 |
| N-acetylarginine | Amino Acid | Urea cycle; Arginine and Proline | HMDB04620 | −0.755 | 0.026 |
| | | Metabolism | | | |
| 4-guanidinobutanoate | Amino Acid | Guanidino and Acetamido Metabolism | HMDB03464 | 0.347 | 0.026 |
| 1-carboxyethylisoleucine | Amino Acid | Leucine, Isoleucine and Valine | N/A | 0.307 | 0.026 |
| | | Metabolism | | | |
| Cysteinylglycine disulfide | Amino Acid | Glutathione Metabolism | HMDB00709 | 1.562 | 0.027 |
| Guanidinoacetate | Amino Acid | Creatine Metabolism | HMDB00128 | −1.125 | 0.027 |
| N2-acetyl,N6-Methyllysine | Amino Acid | Lysine Metabolism | N/A | −0.213 | 0.028 |
| Lysine | Amino Acid | Lysine Metabolism | HMDB00182 | −1.395 | 0.031 |
| 1,6-anhydroglucose | Xenobiotics | Food Component/Plant | HMDB00640 | 0.097 | 0.032 |
| Pyrraline | Xenobiotics | Food Component/Plant | HMDB33143 | 0.190 | 0.032 |
| Mannose | Carbohydrate | Fructose, Mannose and Galactose | HMDB00169 | 0.633 | 0.032 |
| | | Metabolism | | | |
| Ectoine | Xenobiotics | Chemical | N/A | 0.123 | 0.036 |
| 6-bromotryptophan | Amino Acid | Tryptophan Metabolism | N/A | −0.758 | 0.037 |
| 1-linoleoyl-GPA (18:2) | Lipid | Lysophospholipid | HMDB07856 | −0.371 | 0.039 |
| Eicosenoylcarnitine (C20:1) | Lipid | Fatty Acid Metabolism (Acyl Carnitine, | N/A | −0.557 | 0.039 |
| | | Monounsaturated) | | | |
| Erucate (22:1n9) | Lipid | Long Chain Monounsaturated Fatty Acid | HMDB02068 | 0.346 | 0.040 |
| Bilirubin | Cofactors and | Hemoglobin and Porphyrin Metabolism | HMDB00054 | −0.432 | 0.042 |
| | Vitamins | | | | |
| Stearoyl ethanolamide | Lipid | Endocannabinoid | HMDB13078 | 0.070 | 0.043 |
| 3-phenylpropionate (hydrocinnamate) | Xenobiotics | Benzoate Metabolism | HMDB00764 | −0.178 | 0.043 |
| beta-hydroxyisovalerate | Amino Acid | Leucine, Isoleucine and Valine | HMDB00754 | 0.723 | 0.045 |
| | | Metabolism | | | |
| Myo-inositol | Lipid | Inositol Metabolism | HMDB00211 | 0.944 | 0.045 |
| Gulonate | Cofactors and | Ascorbate and Aldarate Metabolism | HMDB03290 | 0.575 | 0.047 |
| | Vitamins | | | | |
| Gluconate | Xenobiotics | Food Component/Plant | HMDB00625 | 0.539 | 0.047 |
| Tryptophan | Amino Acid | Tryptophan Metabolism | HMDB00929 | −1.139 | 0.048 |
| 1-carboxyethylleucine | Amino Acid | Leucine, Isoleucine and Valine | N/A | 0.350 | 0.048 |
| | | Metabolism | | | |
| alpha-ketobutyrate | Amino Acid | Methionine, Cysteine, SAM and Taurine | HMDB00005 | 0.268 | 0.049 |
| | | Metabolism | | | |
| Lanthionine | Amino Acid | Methionine, Cysteine, SAM and Taurine | N/A | −0.229 | 0.049 |
| | | Metabolism | | | |

Commonly Identified Metabolites from Two Different Analytic Approaches

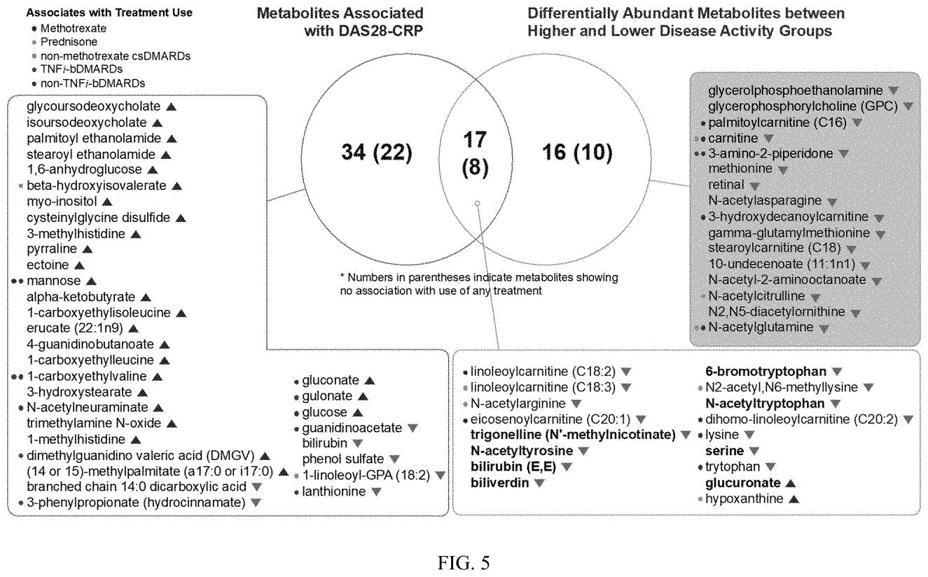

To summarize the findings above, we found that, from the 686 total detectable metabolites in a metabolomic profile, 33 (4.8%) were differentially abundant between higher and lower disease activity; and 51 (7.4%) were significantly associated with DAS28-CRP (FIG. 5). These separate findings amounted to a total of 67 unique metabolites, among which were found to have no association with the use of prednisone, methotrexate, other non-methotrexate csDMARDs, TNFi-bDMARDs, or non-TNFi-bDMARDs. Eight metabolites (6-bromotryptophan, bilirubin (E,E), biliverdin, glucuronate, N-acetyltryptophan, N-acetyltyrosine, serine, and trigonelline) were not only consistently detected across both analytic approaches, but also found to have no association with any treatment use; these results strongly suggest key metabolic pathways and modules potentially contributing to, or serving as indicators of, RA pathogenesis independent of confounding treatment effects. Consistent with this idea, additional studies into the metabolites found in this study (the majority of which have yet to be linked to RA) may be able to provide new insight into the perturbed physiological metabolic processes-which are then in turn reflected in blood underlying disease progression in RA.

Metabolites Associated with CRP Patient Groups

Elevated levels of C-reactive protein (CRP) in the blood is well known to often indicate increased inflammatory conditions, which may be caused by a wide variety of acute (e.g., infections) and chronic disorders (e.g., rheumatoid arthritis, inflammatory bowel disease). In RA patients, CRP levels have been observed to increase after acute mental stress tasks, and also to be linked to risk of cardiovascular disease. Furthermore, several serum metabolites were found to reflect inflammatory activity in patients with early arthritis.

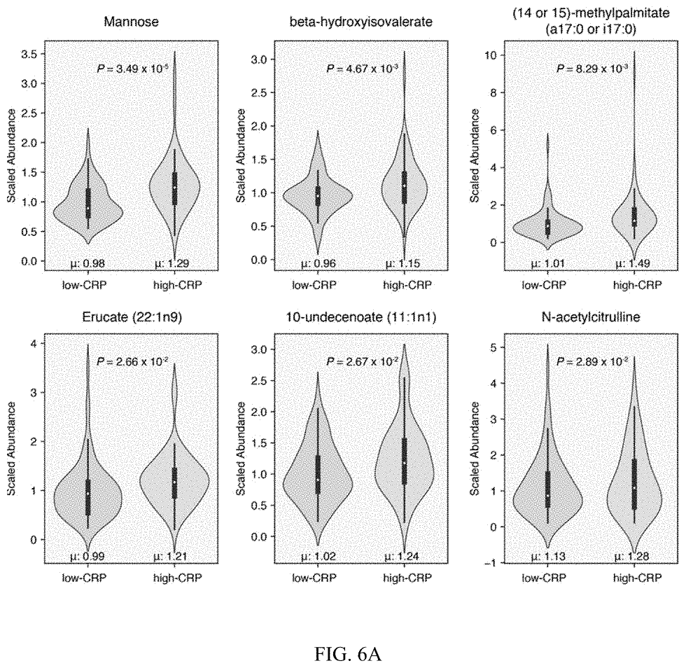

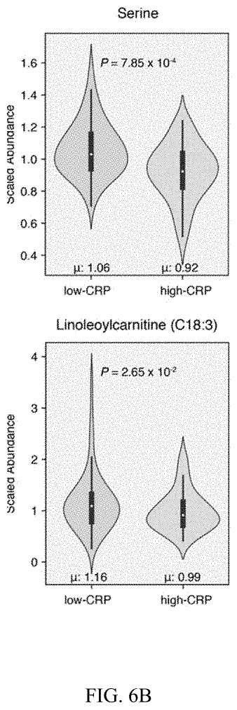

The aforementioned 67 plasma metabolites were further investigated to see whether any were differentially abundant between two CRP patient groups, i.e., ‘high-CRP’ (CRP>3.0 mg/L, n=52) and ‘low-CRP’ (CRP≤3.0 mg/L, n=76). While controlling for potential confounding variables, eight total metabolites were identified that were significantly associated with CRP patient group. More specifically, the abundances of mannose, beta-hydroxyisovalerate, (14 or 15)-methylpalmitate (a17:0 or i17:0), erucate (22:1n9), 10-undecenoate (11:1n1), N-acetylcitrulline were higher in high-CRP, while those of serine and linoleoylcarnitine (C18:3) were lower in high-CRP (FIG. 6). Application of these plasma metabolites, which were found to be connected to both RA disease activity and circulating CRP levels, may lead to the development of new clinical laboratory tests to further enable precision medicine for RA patients.

Plasma Metabolites Associate with Clinical Improvement in RA

Based upon the European League Against Rheumatism (EULAR) response criteria for DAS28-CRP (Wells et al., Annals Rheum. Dis., 68(6):954-960 (2009)), it was found that sixteen of the 64 patients in the discovery cohort showed moderate or good improvement in disease activity from visit 1 to visit 2, while the remaining 48 patients did not show clinical improvement at the time of their second visit. This discovery provided an entry point for the following analysis: For each of these two patient groups, i.e., ‘Improved’ (n=16) and ‘Non-improved’ (n=48) patients, metabolites whose abundances significantly changed from visit 1 to visit 2 were identified while controlling for the same confounding factors (mixed-effects regression model, P<0.05). As a result, eleven metabolites were identified whose abundances significantly changed in the Improved patient group (Table 4), while nineteen metabolites showed significant changes in the Non-improved patient group (Table 5). The following three metabolites, which were discovered in our previous analyses on the 128 plasma metabolome samples of the discovery cohort, were detected once again: erucate (22:1n9), a metabolite identified to be associated with both DAS28-CRP and CRP patient group, was identified to be significantly different between visit 1 and visit 2 in patients who did not show clinical improvement (Non-improved); 3-amino-2-piperidone, a metabolite identified to be differentially abundant between higher and lower disease activity in our study, was identified to be significantly different between visit 1 and visit 2 in patients who showed clinical improvement (Improved); and gamma-glutamylmethionine, a metabolite identified to be differentially abundant between higher and lower disease activity, was identified to be significantly different between visit 1 and visit 2 in the Non-improved group. These results allow us to expand our future direction to investigate metabolites associated with clinical improvement in patients with RA.

TABLE 4
Metabolites displaying significant changes in abundances in patients with clinical improvement (n = 16).

| Chemical | | | | | mean | mean | log₂ |
|---|---|---|---|---|---|---|---|
| ID^{α} | P-value | BIOCHEMICAL | SUPER PATHWAY^{β} | SUB PATHWAY^{β} | (visit 1)^{γ} | (visit 2)^{γ} | fold-change^{δ} |
| 100006620 | 0.0095 | nonanoylcarnitine (C9) | Lipid | Fatty Acid Metabolism (Acyl | 0.758 | 1.525 | 1.009 |
| | | | | Carnitine, Medium Chain) | | | |
| 100021136 | 0.0126 | 3-decenoylcarnitine | Lipid | Fatty Acid Metabolism (Acyl | 4.023 | 2.452 | −0.714 |
| | | | | Carnitine, Monounsaturated) | | | |
| 100001658 | 0.0162 | taurolithocholate 3-sulfate | Lipid | Secondary Bile Acid Metabolism | 0.972 | 1.618 | 0.735 |
| 100001121 | 0.0166 | pyridoxate | Cofactors and | Vitamin B6 Metabolism | 0.817 | 1.348 | 0.722 |
| | | | Vitamins | | | | |
| 100001425 | 0.0169 | 5,6-dihydrouridine | Nucleotide | Pyrimidine Metabolism, Uracil | 0.863 | 1.565 | 0.859 |
| | | | | containing | | | |
| 361 | 0.0191 | inosine | Nucleotide | Purine Metabolism, | 4.470 | 0.809 | −2.466 |
| | | | | (Hypo)Xanthine/Inosine containing | | | |
| 100010896 | 0.0340 | 2′-O-methyluridine | Nucleotide | Pyrimidine Metabolism, Uracil | 0.907 | 1.005 | 0.148 |
| | | | | containing | | | |
| 100020361 | 0.0342 | 3-amino-2-piperidone | Amino Acid | Urea cycle; Arginine and Proline | 4.499 | 0.674 | −2.739 |
| | | | | Metabolism | | | |
| 100001359 | 0.0366 | aconitate [cis or trans] | Energy | TCA Cycle | 1.068 | 1.393 | 0.383 |
| 100004414 | 0.0449 | 2-hydroxyphytanate | Lipid | Fatty Acid, Branched | 0.937 | 1.088 | 0.216 |
| 100000285 | 0.0462 | N-alpha-acetylornithine | Amino Acid | Urea cycle; Arginine and Proline | 0.979 | 1.234 | 0.335 |
| | | | | Metabolism | | | |

^{α}Chemical ID defined by Metabolon’s Discovery HD4 ™ platform
^{β}Super-pathways and sub-pathways were defined by Metabolon’s Discovery HD4 ™ platform
^{γ}Mean abundance of the metabolites
^{δ}fold change = mean (visit 2)/mean (visit 1)

TABLE 5
Metabolites displaying significant changes in abundances in patients without clinical improvement (n = 48).

| Chemical | | | | | mean | mean | log₂ |
|---|---|---|---|---|---|---|---|
| ID^{α} | P-value | BIOCHEMICAL | SUPER PATHWAY^{β} | SUB PATHWAY^{β} | (visit 1)^{γ} | (visit 2)^{γ} | fold-change^{δ} |
| 1221 | 0.0009 | creatine | Amino Acid | Creatine Metabolism | 0.819 | 1.262 | 0.624 |
| 100000054 | 0.0049 | 5-hydroxylysine | Amino Acid | Lysine Metabolism | 0.938 | 1.184 | 0.337 |
| 361 | 0.0151 | inosine | Nucleotide | Purine Metabolism, | 2.662 | 1.463 | −0.863 |
| | | | | (Hypo)Xanthine/Inosine containing | | | |
| 100020487 | 0.017 | N-acetyl-isoputreanine | Amino Acid | Polyamine Metabolism | 0.904 | 1.166 | 0.368 |
| 93 | 0.0255 | alpha-ketoglutarate | Energy | TCA Cycle | 0.975 | 1.064 | 0.126 |
| 100001872 | 0.0275 | 1-stearoyl-2- | Lipid | Phosphatidylserine (PS) | 1.418 | 0.886 | −0.678 |
| | | arachidonoyl-GPS | | | | | |
| | | (18:0/20:4) | | | | | |
| 100009394 | 0.0292 | hexadecadienoate | Lipid | Long Chain Polyunsaturated Fatty | 0.955 | 1.098 | 0.202 |
| | | (16:2n6) | | Acid (n3 and n6) | | | |
| 197 | 0.0299 | S-adenosylhomocysteine | Amino Acid | Methionine, Cysteine, SAM and | 0.828 | 1.054 | 0.348 |
| | | (SAH) | | Taurine Metabolism | | | |
| 100006620 | 0.0333 | nonanoylcarnitine (C9) | Lipid | Fatty Acid Metabolism (Acyl | 0.968 | 1.435 | 0.568 |
| | | | | Carnitine, Medium Chain) | | | |
| 100006438 | 0.0397 | citraconate/glutaconate | Energy | TCA Cycle | 1.374 | 0.998 | −0.462 |
| 100020478 | 0.0408 | dodecadienoate (12:2) | Lipid | Fatty Acid, Dicarboxylate | 0.972 | 1.356 | 0.481 |
| 100001605 | 0.0416 | catechol sulfate | Xenobiotics | Benzoate Metabolism | 1.003 | 1.119 | 0.158 |
| 100005998 | 0.0423 | octadecanedioylcarnitine | Lipid | Fatty Acid Metabolism (Acyl | 1.031 | 1.189 | 0.206 |
| | | (C18-DC) | | Carnitine, Dicarboxylate) | | | |
| 100004396 | 0.0457 | 3-hydroxyadipate | Lipid | Fatty Acid, Dicarboxylate | 1.480 | 0.906 | −0.708 |
| 2054 | 0.0458 | ethylmalonate | Amino Acid | Leucine, Isoleucine and Valine | 0.963 | 1.111 | 0.206 |
| | | | | Metabolism | | | |
| 100020541 | 0.0472 | 11beta- | Lipid | Androgenic Steroids | 1.356 | 0.922 | −0.557 |
| | | hydroxyandrosterone | | | | | |
| | | glucuronide | | | | | |
| 1087 | 0.0484 | erucate (22:1n9) | Lipid | Long Chain Monounsaturated Fatty | 0.869 | 1.158 | 0.414 |
| | | | | Acid | | | |
| 100001313 | 0.0490 | gamma- | Peptide | Gamma-glutamyl Amino Acid | 1.003 | 1.097 | 0.129 |
| | | glutamylmethionine | | | | | |
| 100004414 | 0.0498 | 2-hydroxyphytanate | Lipid | Fatty Acid, Branched | 0.972 | 1.073 | 0.143 |

^{α}Chemical ID defined by Metabolon’s Discovery HD4 ™ platform
^{β}Super-pathways and sub-pathways were defined by Metabolon's Discovery HD4 ™ platform
^{γ}Mean abundance of the metabolites
^{δ}fold change = mean (visit 2)/mean (visit 1)

Together these results demonstrate that circulating metabolites can be used to identify a mammal (e.g., a human) has having RA. For example, a distinct metabolite signature present in a blood sample obtained from a human can be used to identify that human as having RA. In some cases, the distinct metabolite signature can be used to determine that activity and/or disease stage of the RA.

### Example 2: A Generalized Linear Model Predicts DAS28-CRP Score

A mixed-effects linear regression model was used on 128 RA plasma samples to select metabolites that were significantly associated with DAS28-CRP. As a result, 51 metabolites were identified (P<0.05), which were then used as predictor variables for a machine-learning model (i.e., generalized linear model [GLM]) for DAS28-CRP estimation. The main components of the GLM, that is, the coefficients and intercept, were estimated based on the 128 RA plasma samples. Below, Equation (1) summarizes the final constructed GLM for DAS28-CRP prediction based on the abundance of 51 metabolites. Mᵢ represents the abundance of metabolite i in the patient's plasma sample, while βᵢ is the coefficient corresponding to Mᵢ. For example, M₁ is the measured (scaled) abundance of glycoursodeoxycholate, whose model coefficient β₁ is −0.0137707. Table 6 summarizes the corresponding coefficients (βᵢ) of each metabolite.

$${DAS28\_CRP \sum }_{i = 1}^{E 1} ( {β}_{i} {M}_{i} ) + 2.641856 \qquad ( 1 )$$

TABLE 6
Plasma metabolites significantly associated with DAS28-CRP.

| Metabolite | | Coefficient | |
|---|---|---|---|
| index | Metabolite (Mᵢ) | index | Coefficient (βᵢ) |
| M₁ | Glycoursodeoxycholate | β₁ | −0.0137707 |
| M₂ | Eicosenoylcarnitine (C20:1) | β₂ | −0.829059 |
| M₃ | Branched chain 14:0 | β₃ | −0.1291121 |
| | dicarboxylic acid | | |
| M₄ | Lysine | β₄ | −0.2994297 |
| M₅ | 3-methylhistidine | β₅ | 0.12286813 |
| M₆ | 1-carboxyethylleucine | β₆ | −0.6156973 |
| M₇ | Biliverdin | β₇ | −0.1344605 |
| M₈ | Trigonelline (N′- | β₈ | −0.1354869 |
| | methylnicotinate) | | |
| M₉ | Bilirubin | β₉ | 0.08899905 |
| M₁₀ | Isoursodeoxycholate | β₁₀ | 0.02911994 |
| M₁₁ | Glucose | β₁₁ | −0.5245139 |
| M₁₂ | 1-carboxyethylisoleucine | β₁₂ | −0.5939626 |
| M₁₃ | 3-phenylpropionate | β₁₃ | −0.0691645 |
| | (hydrocinnamate) | | |
| M₁₄ | Palmitoyl ethanolamide | β₁₄ | 0.08090326 |
| M₁₅ | Tryptophan | β₁₅ | 0.83265799 |
| M₁₆ | Dimethylguanidino valeric | β₁₆ | 0.24035438 |
| | acid (DMGV) | | |
| M₁₇ | Guanidinoacetate | β₁₇ | −0.147475 |
| M₁₈ | Phenol sulfate | β₁₈ | −0.1682771 |
| M₁₉ | Cysteinylglycine disulfide | β₁₉ | 1.23610258 |
| M₂₀ | Linoleoylcarnitine (C18:3) | β₂₀ | −1.0951146 |
| M₂₁ | 1-linoleoyl-GPA (18:2) | β₂₁ | −0.1236492 |
| M₂₂ | Hypoxanthine | β₂₂ | 0.25248128 |
| M₂₃ | Mannose | β₂₃ | −0.0458874 |
| M₂₄ | Pyrraline | β₂₄ | 0.1463747 |
| M₂₅ | Ectoine | β₂₅ | 0.03377761 |
| M₂₆ | Trimethylamine N-oxide | β₂₆ | −0.1108116 |
| M₂₇ | N2-acetyl,N6-methyllysine | β₂₇ | −0.0043281 |
| M₂₈ | beta-hydroxyisovalerate | β₂₈ | −0.1330342 |
| M₂₉ | N-acetylarginine | β₂₉ | −0.096799 |
| M₃₀ | Stearoyl ethanolamide | β₃₀ | −0.0712158 |
| M₃₁ | Glucuronate | β₃₁ | 0.35355433 |
| M₃₂ | 6-bromotryptophan | β₃₂ | −0.4218997 |
| M₃₃ | Bilirubin (E,E) | β₃₃ | −0.3167004 |
| M₃₄ | N-acetyltyrosine | β₃₄ | −0.748858 |
| M₃₅ | Gluconate | β₃₅ | 0.98991591 |
| M₃₆ | 1-methylhistidine | β₃₆ | 0.67179543 |
| M₃₇ | 1,6-anhydroglucose | β₃₇ | −0.0045716 |
| M₃₈ | (14 or 15)-methylpalmitate | β₃₈ | 0.20117096 |
| | (a17:0 or i17:0) | | |
| M₃₉ | 4-guanidinobutanoate | β₃₉ | 0.07228207 |
| M₄₀ | N-acetylneuraminate | β₄₀ | 0.43856713 |
| M₄₁ | Dihomo-linoleoylcarnitine | β₄₁ | −0.4122119 |
| | (C20:2) | | |
| M₄₂ | Erucate (22:1n9) | β₄₂ | 0.2423382 |
| M₄₃ | 1-carboxyethylvaline | β₄₃ | 1.23402343 |
| M₄₄ | Serine | β₄₄ | −0.8581117 |
| M₄₅ | Lanthionine | β₄₅ | −0.0125453 |
| M₄₆ | alpha-ketobutyrate | β₄₆ | −0.1686771 |
| M₄₇ | Myo-inositol | β₄₇ | 0.57921768 |
| M₄₈ | N-acetyltryptophan | β₄₈ | −0.2090842 |
| M₄₉ | Gulonate | β₄₉ | −1.2903587 |
| M₅₀ | Linoleoylcarnitine (C18:2) | β₅₀ | 2.12785351 |
| M₅₁ | 3-hydroxystearate | β₅₁ | 0.1620114 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-13. (canceled)

14. A method for treating a mammal having arthritis, wherein said method comprises:
(a) determining that a blood sample from said mammal comprises a low disease activity signature, and
(b) administering an arthritis drug to said mammal,
wherein said low disease activity signature comprises (1a) an increased level of five or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of five or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate.

15. The method of claim 14, wherein said mammal is a human.

16. The method of claim 14, wherein said arthritis is a rheumatoid arthritis.

17. The method of claim 14, wherein said blood sample is a plasma sample.

18. The method of claim 14, wherein said low disease activity signature comprises (1a) an increased level of 10 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of 10 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate.

19. The method of claim 14, wherein said low disease activity signature comprises (1a) an increased level of 15 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of 15 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate.

20. The method of claim 14, wherein said low disease activity signature comprises (1a) an increased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol or (1b) a decreased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate.

21. The method of claim 14, wherein said low disease activity signature comprises (1a) an increased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol and (1b) a decreased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate.

22. The method of claim 14, wherein said arthritis drug is selected from the group consisting of methotrexate, hydroxychloroquine, sulfasalazine, and leflunomide.

23-30. (canceled)

31. A method for treating a mammal having arthritis, wherein said method comprises:
(a) determining that a blood sample from said mammal comprises a moderate-to-high disease activity signature, and
(b) administering an arthritis drug to said mammal or performing surgery to treat said arthritis,
wherein said moderate-to-high disease signature comprises (2a) an increased level of five or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of five or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol.

32. The method of claim 31, wherein said mammal is a human.

33. The method of claim 31, wherein said arthritis is a rheumatoid arthritis.

34. The method of claim 31, wherein said blood sample is a plasma sample.

35. The method of claim 31, wherein said moderate-to-high disease signature comprises (2a) an increased level of 10 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of 10 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol.

36. The method of claim 31, wherein said moderate-to-high disease signature comprises (2a) an increased level of 15 or more metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of 15 or more metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol.

37. The method of claim 31, wherein said moderate-to-high disease signature comprises (2a) an increased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate or (2b) a decreased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol.

38. The method of claim 31, wherein said moderate-to-high disease signature comprises (2a) an increased level of the metabolites selected from the group consisting of (14 or 15)-methylpalmitate (a17:0 or i17:0), 1,6-anhydroglucose, N-acetylneuraminate, hypoxanthine, 1-carboxyethylleucine, ectoine, pyrraline, cysteinylglycine disulfide, erucate (22:1n9), 3-methylhistidine, mannose, dimethylguanidino valeric acid (DMGV), 1-carboxyethylvaline, beta-hydroxyisovalerate, stearoyl ethanolamide, trimethylamine N-oxide, 3-hydroxystearate, gluconate, palmitoyl ethanolamide, glucose, and glucoronate and (2b) a decreased level of the metabolites selected from the group consisting of isoursodeoxycholate, linoleoylcarnitine (C18:2), dihomo-linoleoylcarnitine (C20:2), N-acetyltyrosine, 1-methylhistidine, 4-guanidinobutanoate, lysine, serine, N-acetyltryptophan, 6-bromotryptophan, 1-carboxyethylisoleucine, alpha-ketobutyrate, N2-acetyl,N6-methyllysine, trigonelline (N′-methylnicotinate), 3-phenylpropionate (hydrocinnamate), tryptophan, N-acetylarginine, 1-linoleoyl-GPA (18:2), gulonate, phenol sulfate, branched chain 14:0 dicarboxylic acid, bilirubin, linoleoylcarnitine (C18:3), bilirubin (E,E), eicosenoylcarnitine (C20:1), lanthionine, glycoursodeoxycholate, biliverdin, guanidinoacetate, and myo-inositol.

39. The method of claim 31, wherein said method comprises administering said arthritis drug to said mammal.

40. The method of claim 39, wherein said arthritis drug is selected from the group consisting of adalimumab, certolizumab, etanercept, golimumab, infliximab, abatacept, tocilizumab, sarilumab, rituximab, tofacitinib, baricitinib, and upadacitinib.

41. The method of claim 31, wherein said method comprises performing said surgery.

42-51. (canceled)
